(19) United States
(12) Patent Application Publication
(10) Pub. No.: US 2024/0398941 A1
(43) Pub. Date: Dec. 5, 2024

(54) LIPID NANOPARTICLE COMPRISING A NUCLEIC ACID-BINDING PROTEIN

(71) Applicant: Seqirus Inc., Holly Springs, NC (US)

(72) Inventors: Steven ROCKMAN, Holly Springs, NC (US); Chi ONG, Holly Springs, NC (US)

(21) Appl. No.: 18/699,125

(22) PCT Filed: Oct. 6, 2022
(86) PCT No.: PCT/IB2022/059527

§ 371 Date: Apr. 5, 2024

(30) Foreign Application Priority Data
Oct. 6, 2021 (AU) 2021903192

(51) Int. Cl. A61K 39/385; A61K 9/51; A61K 39/00; A61K 39/12; A61K 39/145; A61K 39/155; A61K 39/215; A61K 39/235; C12N 15/88

(52) U.S. Cl. CPC A61K 39/385; A61K 9/5123; A61K 39/12; A61K 39/145; A61K 39/155; A61K 39/215; A61K 39/235; C12N 15/88; A61K 2039/53; A61K 2039/6031

(57) ABSTRACT

The present disclosure relates to lipid nanoparticles for delivery of RNA, the lipid nanoparticle comprising therein a nucleic acid-binding protein or peptide (e.g., a RNA-binding protein or peptide) bound to the RNA, and uses thereof.

### RELATED APPLICATION DATA

The present application claims priority from Australian Patent Application No. 2021903192 filed 6 Oct. 2021 entitled “Lipid nanoparticle comprising a RNA-binding protein”, the entire contents of which is hereby incorporated by reference.

### SEQUENCE LISTING

The present application is filed together with a Sequence Listing in electronic format. The entire contents of the Sequence Listing are hereby incorporated by reference.

### FIELD

The present disclosure relates to lipid nanoparticles for delivery of RNA, the lipid nanoparticle comprising therein a nucleic acid-binding protein or peptide (e.g., a RNA-binding protein) bound to the RNA, and uses thereof.

### BACKGROUND

Nucleic acid vaccines have recently emerged as a promising approach to the treatment and prevention of various diseases, including against the severe acute respiratory syndrome coronavirus 2 (SARS-CoV-2) responsible for causing the on-going worldwide pandemic of the severely infectious coronavirus disease 2019 (COVID-19).

mRNA vaccines rely on the delivery of the mRNA into the cytoplasm of host cells, where it is transcribed into antigenic proteins to trigger the production of neutralizing antibodies. However, the large size and negative charge of mRNA prevent cellular uptake. Therefore, lipid delivery vehicles, such as liposomes or lipid nanoparticles are used to encapsulate the mRNA, blocking degradation of the RNA in plasma, whilst also promoting cellular uptake for efficient delivery of the mRNA in vivo.

Lipid delivery vehicles are commonly formed from cationic lipids and other ionisable lipid components such as ionisable lipids, neutral lipids, cholesterol and PEGylated lipids. Cationic lipids are amphiphilic molecules having a lipophilic region containing one or more hydrocarbon groups and a hydrophilic region containing at least one positively charged polar head group. Cationic lipids and nucleic acids form a positively charged complex, making it easier for the nucleic acids to pass through the plasma membrane of the cell and enter the cytoplasm.

However, several adverse cytotoxic effects of cationic lipids are known including, production of reactive oxygen species and accumulation in plasma due to poor degradation by humans. Thus, much effort has focused on identifying novel lipids or particular lipid compositions that can protect nucleic acids from degradation and elimination, as well as provide effective intracellular delivery and/or expression/translation of mRNA. In addition, these lipid-nucleic acid particles must be well tolerated and provide an appropriate therapeutic index so that treatment with an effective dose of nucleic acid must not be associated with unacceptable toxicity and/or risk to the patient.

Therefore, it will be apparent to the skilled person that there is an on-going need for improved lipid nanoparticles for delivery of oligonucleotides, such as RNA.

### SUMMARY

The present disclosure is based on the inventors' finding that incorporation of a RNA-binding protein or peptide or a lipidated RNA-binding protein or peptide in a lipid nanoparticle increases stability of the associated RNA and/or facilitates nucleation of the lipid nanoparticle and/or reduces the toxicity and/or adverse side effects of the lipid nanoparticle. The inventors have also identified that incorporation of the RNA-binding protein or peptide into the lipid nanoparticle is able to protect against toll-like receptor (TLR) stimulation/induction. The inventors have further identified that incorporation of a nucleic acid-binding protein or peptide (i.e., a RNA- and DNA-binding protein) into the lipid nanoparticle is protective.

Broadly, the findings by the inventors provide the basis for a lipid nanoparticle comprising a nucleic acid-binding protein or peptide. The findings by the inventors also provide the basis for a lipid nanoparticle comprising a lipidated nucleic-acid binding protein or peptide.

In one example, the nucleic acid-binding protein or peptide is a RNA-binding protein or peptide. In one example, the nucleic acid-binding protein or peptide is a RNA- and DNA-binding protein.

The findings of the inventors provide the basis for a lipid nanoparticle comprising a RNA-binding protein or peptide. The findings by the inventors further provide the basis for a lipid nanoparticle comprising a lipidated nucleic acid-binding protein or peptide. The findings by the inventors also provide the basis for a lipid nanoparticle comprising a lipidated RNA-binding protein or peptide.

Furthermore, the findings by the inventors provide the basis for methods of use of the lipid nanoparticle as a vaccine or as a therapeutic.

Accordingly, the present disclosure provides a lipid nanoparticle for delivery of RNA (e.g., mRNA), the lipid nanoparticle comprising therein a nucleic acid-binding protein or peptide bound to the RNA.

Accordingly, the present disclosure provides a lipid nanoparticle for delivery of RNA (e.g., mRNA), the lipid nanoparticle comprising therein a RNA-binding protein or peptide bound to the RNA.

In one example, the nucleic acid-binding protein or peptide is a lipidated nucleic acid-binding protein or peptide. In one example, the RNA-binding protein or peptide is a lipidated RNA-binding protein or peptide.

Accordingly, the present disclosure provides a lipid nanoparticle for delivery of RNA (e.g., mRNA), the lipid nanoparticle comprising therein a lipidated nucleic acid-binding protein or peptide bound to the RNA.

Accordingly, the present disclosure provides a lipid nanoparticle for delivery of RNA (e.g., mRNA), the lipid nanoparticle comprising therein a lipidated RNA-binding protein or peptide bound to the RNA.

In one example, the RNA-binding protein or peptide is lipidated prior to binding the RNA. In another example, the RNA-binding protein or peptide is lipidated after binding to the RNA.

In one example, the RNA-binding protein or peptide is lipidated with a lipid moiety selected from the group consisting of a fatty acid, an isoprenoid and combinations thereof.

In one example, the RNA-binding protein or peptide is lipidated with a fatty acid. For example, the fatty acid is a triglyceride, a phospholipid or a cholesteryl ester. In one example, the fatty acid is a triglyceride. In another example, the fatty acid is a phospholipid. In a further example, the fatty acid is a cholesteryl ester.

In one example, the RNA-binding protein or peptide is lipidated with an isoprenoid. For example, the isoprenoid is isoprene.

In one example, the RNA-binding protein or peptide is lipidated on a nucleophilic side chain, at the N-terminal end and/or at the C-terminal end.

In one example, the RNA-binding protein or peptide is lipidated on a nucleophilic side chain. For example, on a cysteine, a serine, a threonine, a tyrosine and/or a lysine amino acid residue. In one example, the nucleophilic side chain is a cysteine residue. In another example, the nucleophilic side chain is a serine residue. In a further example, the nucleophilic side chain is a threonine residue. In one example, the nucleophilic side chain is a tyrosine residue. In another example, the nucleophilic side chain is a lysine residue.

In one example, the RNA-binding protein or peptide is lipidated at the N-terminal end of the protein or peptide.

It will be apparent to the skilled person that the N-terminal end of the RNA-binding protein or peptide comprises a nuclear localisation signal(s) (or sequence) and/or a nuclear export signal. In one example, the RNA-binding protein or peptide is modified to remove nuclear localisation signal(s) and/or introduce nuclear export signal(s).

In one example, RNA-binding protein or peptide is modified to remove the nuclear localisation signal. In another example, the RNA-binding protein or peptide is modified to inactivate or remove nuclear localisation signal(s). For example, the RNA-binding protein or peptide does not comprise a nuclear localisation signal(s) (or sequence). The skilled person will understand that the nuclear localisation signal is one or more sequences of positively charged lysines or arginines exposed on the protein surface that tag a protein for import into the cell nuclear by nuclear transport. Methods of modifying the nuclear localisation signal will be apparent to the skilled person and/or are described herein. For example, the nuclear localisation signal is lipidated or removed or inactivated. In one example, the nuclear localisation signal at the N-terminal end of the RNA-binding protein or peptide is lipidated. In another example, the nuclear localisation signal at the N-terminal end of the RNA-binding protein or peptide is removed. In another example, the nuclear localisation signal at the N-terminal end of the RNA-binding protein or peptide is inactivated.

In one example, the RNA-binding protein or peptide is modified to introduce a nuclear export signal. The skilled person will understand that the nuclear export signal is a short leucine-rich motif that targets the protein for export from the cell nuclear to the cytoplasm through the nuclear pore complex. Methods for introducing a nuclear export signal will be apparent to the skilled person and/or are described herein.

In one example, the RNA-binding protein or peptide is lipidated at the C-terminal end of the protein or peptide.

In one example, the RNA-binding protein or peptide is lipidated by palmitoylation, myristoylation, fatty-acylation, esterification, prenylation or combinations thereof.

In one example, the RNA-binding protein or peptide is lipidated by palmitoylation. For example, N-terminal cysteine palmitoylation.

In one example, the RNA-binding protein or peptide is lipidated by myristoylation. For example, N-terminal glycine myristoylation.

In one example, the RNA-binding protein or peptide is lipidated by fatty-acylation. For example, lysine N-acylation. In another example, serine O-acylation.

In one example, the RNA-binding protein or peptide is lipidated by esterification. For example, C-terminal cholesterol esterification.

In one example, the RNA-binding protein or peptide is lipidated by prenylation. For example, the prenylation is farnesylation or geranylgeranylation. In one example, the prenylation is cysteine prenylation.

In one example, the RNA-binding protein or peptide is lipidated by N-terminal cysteine palmitoylation, N-terminal glycine myristoylation, lysine N-acylation, C-terminal cholesterol esterification, cysteine prenylation, serine O-acylation or combinations thereof.

In one example, the lipid moiety is linked to the RNA-binding protein or peptide by a thioether bond, an ester bond, a thioester bond and/or an amide bond.

In one example, the lipid moiety is linked to the RNA-binding protein or peptide by a thioether bond.

In one example, the lipid moiety is linked to the RNA-binding protein or peptide by an ester bond.

In one example, the lipid moiety is linked to the RNA-binding protein or peptide by a thioester bond.

In one example, the lipid moiety is linked to the RNA-binding protein or peptide by an amide bond.

In one example, the RNA-binding protein or peptide is lipidated using chemical or enzymatic lipidation. For example, the RNA-binding protein or peptide is lipidated using chemical lipidation. In one example, the chemical lipidation is selected from the group consisting of chemical ligation, click chemistry, expressed protein ligation and combinations thereof. In another example, the RNA-binding protein or peptide is lipidated using enzymatic lipidation. For example, the enzymatic lipidation is selected from the group consisting of Sortase-A mediated lipidation, transglutaminase mediated lipidation and combinations thereof. In one example, the enzymatic lipidation is performed in vivo or in vitro. For example, the enzymatic lipidation is performed in vivo. In another example, the enzymatic lipidation is performed in vitro.

In one example, the nucleic acid-binding protein or peptide binds directly to the RNA. In another example, the nucleic acid-binding protein or peptide binds the RNA prior to formulating the RNA into a lipid nanoparticle. In a further example, the nucleic acid-binding protein or peptide binds the RNA in the lipid nanoparticle after formulating the RNA into a lipid nanoparticle, wherein the nucleic acid-binding protein or peptide is within the lipid nanoparticle. For example, the nucleic acid-binding protein or peptide binds the lipid nanoparticle encapsulated RNA.

In one example, the nucleic acid-binding protein or peptide additionally binds to RNA on the surface of the lipid nanoparticle. In such a situation, nucleic acid-binding protein or peptide will be present within the lipid nanoparticle and on the surface of the lipid nanoparticle. The nucleic acid-binding protein or peptide on the surface of the lipid nanoparticle need not be the same as the RNA-binding protein or peptide within the lipid nanoparticle.

For example, a lipid nanoparticle can be formed with a nucleic acid-binding protein or peptide bound to RNA therein and the formed lipid nanoparticle can then be coated with a nucleic acid-binding protein or peptide to bind to any unencapsulated and/or partially encapsulated RNA.

In one example, the nucleic acid-binding protein or peptide encapsulates the RNA. In another example, the nucleic acid-binding protein or peptide binds on a nucleophilic side chain, at the N-terminal end and/or at the C-terminal end of the RNA. In one example, the nucleic acid-binding protein or peptide binds on a nucleophilic side chain of the RNA. In another example, the nucleic acid-binding protein or peptide binds at the N-terminal end and/or at the C-terminal end of the RNA. For example, at the N-terminal end of the RNA. In another example, at the C-terminal end of the RNA. For example, the nucleic acid-binding protein or peptide does not encapsulate the RNA.

In one example, the RNA-binding protein or peptide binds directly to the RNA.

In another example, the RNA-binding protein or peptide binds the RNA prior to formulating the RNA into a lipid nanoparticle. In a further example, the RNA-binding protein or peptide binds the RNA in the lipid nanoparticle after formulating the RNA into a lipid nanoparticle, wherein the RNA-binding protein or peptide is within the lipid nanoparticle. For example, the RNA-binding protein or peptide binds the lipid nanoparticle encapsulated RNA.

In one example, the RNA-binding protein or peptide additionally binds to RNA on the surface of the lipid nanoparticle. In such a situation, RNA-binding protein or peptide will be present within the lipid nanoparticle and on the surface of the lipid nanoparticle. The RNA-binding protein or peptide on the surface of the lipid nanoparticle need not be the same as the RNA-binding protein or peptide within the lipid nanoparticle.

For example, a lipid nanoparticle can be formed with a RNA-binding protein or peptide bound to RNA therein and the formed lipid nanoparticle can then be coated with a RNA-binding protein or peptide to bind to any unencapsulated and/or partially encapsulated RNA.

In one example, the RNA-binding protein or peptide encapsulates the RNA. In another example, the RNA-binding protein or peptide binds on a nucleophilic side chain, at the N-terminal end and/or at the C-terminal end of the RNA. In one example, the RNA-binding protein or peptide binds on a nucleophilic side chain of the RNA. In another example, the RNA-binding protein or peptide binds at the N-terminal end and/or at the C-terminal end of the RNA. For example, at the N-terminal end of the RNA. In another example, at the C-terminal end of the RNA. For example, the RNA-binding protein or peptide does not encapsulate the RNA.

In one example, the nucleic acid-binding protein or peptide:
a) reduces toxicity of the lipid nanoparticle,
b) stabilizes the RNA,
c) protects the RNA from degradation,
d) facilitates nucleation of the lipid nanoparticle, and/or
e) inhibits induction of signalling by one or more Toll-like receptors.

In one example, the RNA-binding protein or peptide:
a) reduces toxicity of the lipid nanoparticle,
b) stabilizes the RNA,
c) protects the RNA from degradation,
d) facilitates nucleation of the lipid nanoparticle, and/or
e) inhibits induction of signalling by one or more Toll-like receptors.

In one example, the RNA-binding protein or peptide reduces toxicity of the lipid nanoparticle.

In one example, the RNA-binding protein or peptide stabilizes the RNA.

In one example, the RNA-binding protein or peptide protects the RNA from degradation.

In one example, the RNA-binding protein or peptide facilitates nucleation of the lipid nanoparticle.

In one example, the RNA-binding protein or peptide inhibits induction of signalling by one or more Toll-like receptors. In one example, the RNA-binding protein or peptide does not inhibit induction of signalling by one or more Toll-like receptors.

The skilled person will understand that there are a set of Toll-like receptors, namely endosomal Toll-like receptors comprising TLR3, TLR7, TLR8 and TLR9, that recognise and bind nucleic acids, such as RNA. Activation of these receptors leads to production of inflammatory cytokines, as well as type I interferons (interferon type I).

In one example, the RNA-binding protein or peptide inhibits induction of signalling by one or more endosomal Toll-like receptors. For example, the RNA-binding protein or peptide inhibits induction of signalling by one or more Toll-like receptors selected from the group consisting of TLR3, TLR7, TLR8 and TLR9. In one example, the RNA-binding protein or peptide inhibits induction of signalling by TLR3. In another example, the RNA-binding protein or peptide inhibits induction of signalling by TLR7/9. In a further example, the RNA-binding protein or peptide inhibits induction of signalling by TLR8.

In one example, the RNA-binding protein or peptide is two RNA-binding proteins or peptides (i.e., a first and a second RNA-binding protein or peptide) linked by a linker. For example, the first and second RNA-binding proteins or peptides are covalently linked by an amide bond. The present disclosure encompasses other forms of covalent and non-covalent linkages. For example, the RNA-binding proteins or peptides can be linked by a chemical linker.

In one example, the linker is a flexible linker, e.g., a flexible peptide linker. For example, the first RNA-binding protein or peptide is linked to the second RNA-binding protein via a flexible linker.

In one example, the linker is a peptide linker. For example, the first RNA-binding protein or peptide is linked to the second RNA-binding protein or peptide via a linker wherein the linker is a peptide linker comprising between 2 and 31 amino acids in length. In one example, the linker comprises the sequence (Gly₄Ser)ₙ, wherein n is between 1 and 6. For example, the linker comprises the sequence SGGGGS (GS6) or the sequence SGGGGSGGGGSGGGGSGGGGSGGGGSGGGGS (GS31). In another example, the linker comprises the sequence (Ala) n, wherein n is between 2 and 31.

In one example, the linker is a rigid linker. For example, the rigid linker comprises the sequence (EAAAK) n, where n is between 1 and 3. In one example, the rigid linker comprises the (EAAAK) n, where n is between 1 and 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 60, or less than about 50, or less than about 40, or less than about 30, or less than about 20, or less than about 10.

In one example, the nucleic acid-binding protein or peptide is a viral or non-viral nucleic acid-binding protein or peptide. For example, the nucleic acid-binding protein is a viral nucleic acid-binding protein. In another example, the nucleic acid-binding protein is a non-viral nucleic acid binding protein.

In one example, the RNA-binding protein or peptide is a viral or non-viral RNA-binding protein or peptide.

In one example, the RNA-binding protein or peptide is a viral RNA-binding protein. For example, the viral RNA-binding protein or peptide is from a class III, class IV, class V and/or class IV virus. In one example, the viral RNA-binding protein or peptide is from a class III virus. In another example, the viral RNA-binding protein or peptide is from a class IV virus. In a further example, the viral RNA-binding protein or peptide is from a class V virus. In one example, the RNA-binding protein or peptide is from a class VI virus.

In one example, the viral RNA-binding protein or peptide is from a respiratory virus selected from the group consisting of an influenza virus, a respiratory syncytial virus, a parainfluenza virus, a metapneumovirus, a rhinovirus, a coronavirus, an adenovirus and a bocavirus.

In one example, the viral RNA-binding protein or peptide is from an influenza virus. For example, the influenza virus is influenza A. In another example, the influenza virus is influenza B.

In one example, the viral RNA-binding protein or peptide is from a respiratory syncytial virus.

In one example, the viral RNA-binding protein or peptide is from a parainfluenza virus.

In one example, the viral RNA-binding protein or peptide is from a metapneumovirus.

In one example, the viral RNA-binding protein or peptide is from a rhinovirus.

In one example, the viral RNA-binding protein or peptide is from a coronavirus. For example, the coronavirus is severe acute respiratory disease 2 (SARS-COV 2).

In one example, the viral RNA-binding protein or peptide is from an adenovirus.

In one example, the viral RNA-binding protein or peptide is from a bocavirus.

In one example, the viral RNA-binding protein or peptide is a nucleoprotein, a non-structural protein, a matrix protein and/or a nucleocapsid protein. For example, the viral RNA-binding protein or peptide is a nucleoprotein. In another example, the viral RNA-binding protein or peptide is a matrix protein. In a further example, the viral RNA-binding protein or peptide is a nucleoprotein. In another example, the viral RNA-binding protein or peptide is a non-structural protein.

In one example, the viral RNA-binding protein or peptide comprises a sequence set forth in any one of SEQ ID NOs 9 to 11.

In one example, the viral RNA-binding protein or peptide is a non-structural (NS) protein from an influenza B virus. For example, the viral RNA-binding protein or peptide is an influenza B NS1 RNA binding domain (RBD). In one example, the viral RNA-binding protein or peptide is influenza B NS1 RBDA. In another example, the viral RNA-binding protein or peptide is influenza B NS1 RBDB. In yet another example, the viral RNA-binding protein or peptide is influenza B NS1 RBDC. In one example, the influenza B NS1 RNA binding domain is a full length binding domain. In another example, the influenza B NS1 RNA binding domain is a truncated binding domain. In a further example, the influenza B NS1 RNA binding domain is a modified binding domain. In one example, the influenza B NS1 RNA binding domain is set forth in SEQ ID NO: 9. In another example, the influenza B NS1 RNA binding domain is set forth in SEQ ID NO: 10. In a further example, the influenza B NS1 RNA binding domain is set forth in SEQ ID NO: 11. In one example, the influenza B NS1 RNA binding domain is a modified binding domain comprising a first influenza B NS1 RNA binding domain set forth in SEQ ID NO: 11 and a second influenza B NS1 RNA binding domain set forth in SEQ ID NO: 10, wherein the first and second RNA binding domains are linked by a suitable linker. For example, the 3′ end of the first influenza B NS1 RNA binding domain is linked to the 5′ end of the second influenza B NS1 RNA binding domain.

In one example, the viral nucleic acid-binding protein is from a hepadnavirus. For example, the hepadnavirus is hepatitis B virus (HBV).

In one example, the viral RNA-binding protein or peptide is a nucleoprotein, wherein the RNA-binding protein or peptide encapsulates the RNA, stabilizes the RNA and inhibits induction of signalling by one or more endosomal Toll-like receptors (e.g., TLR3, TLR7, TLR8 and/or TLR9).

In one example, the viral RNA-binding protein or peptide is a nucleocapsid, wherein the RNA-binding protein or peptide encapsulates the RNA, stabilizes the RNA and inhibits induction of signalling by one or more endosomal Toll-like receptors (e.g., TLR3, TLR7, TLR8 and/or TLR9).

In one example, the viral RNA-binding protein or peptide is a matrix protein, wherein the RNA-binding protein or peptide binds to the RNA, stabilizes the RNA, but does not inhibit induction of signalling by one or more endosomal Toll-like receptors (e.g., TLR3, TLR7, TLR8 and/or TLR9).

In one example, the RNA-binding protein or peptide is a non-viral RNA-binding protein or peptide. For example, the RNA-binding protein or peptide is a non-viral protein or peptide derived from cellular proteins. In one example, the RNA-binding protein or peptide is derived from cellular proteins associated with cell growth, cell signalling and/or anti-viral pathways.

In one example, the cellular protein is selected from the group consisting of a TAR RNA binding protein (TRBP), a protein kinase R (PKR) RNA binding protein, a Toll-like Receptor 3 (TLR-3) binding protein, a TLR-7 binding protein and combinations thereof.

In one example, the cellular protein comprises a sequence set forth in any one of SEQ ID NOs: 1 to 8.

In one example, the cellular protein is a TAR RNA binding protein (TRBP). For example, the cellular protein is TRBP RNA binding domain (RBD) 2. In one example, the cellular protein is TRBP RBDA. In another example, the cellular protein is TRBP RBDB. In one example, the TRBP RNA binding domain 2 is full length. For example, the full length TRBP RNA binding domain 2 is set forth in SEQ ID NO: 1. In another example, the TRBP RNA binding domain 2 is a truncated binding domain. For example, the truncated TRBP RNA binding domain 2 is set forth in SEQ ID NO: 2.

In one example, the cellular protein is a protein kinase R (PKR) RNA-binding protein. For example, the cellular protein is PKR RNA-binding motif 2. In one example, the cellular protein is PKR RNA-binding domain (RBD). In another example, the cellular protein is PKR RBDA. In a further example, the cellular protein is PKR RBDB. In one example, the PKR RNA-binding motif 2 is a full length binding motif. For example, the full length PKR RNA-binding motif 2 is set forth in SEQ ID NO: 3. In another example, the PKR RNA-binding motif 2 is a truncated binding motif. For example, the truncated PKR RNA-binding motif 2 is set forth in SEQ ID NO: 4. In another example, the truncated PKR RNA-binding motif 2 is set forth in SEQ ID NO: 5.

In one example, the cellular protein is a TLR-3 dsRNA-binding domain 1. For example, the cellular protein is a TLR-3 dsRNA-binding domain 1 (leucine rich repeats 1-3). In another example, the cellular protein is a TLR-3 dsRNA-binding domain 1 (leucine rich repeats 17-18). In one example, the TLR-3 dsRNA-binding domain 1 is set forth in SEQ ID NO: 6. In a further example, the TLR-3 dsRNA-binding domain 1 is set forth in SEQ ID NO: 7. In another example, the TLR-3 is TLR-3 leucine-rich repeat (LRR) A. In yet another example, the TLR-3 is TLR-3 LRRB.

In one example, the cellular protein is a TLR-7 RNA-binding site. For example, the cellular protein is a TLR-7 RNA-binding site (leucine rich repeats 14-15). In one example, the TLR-7 RNA-binding site is set forth in SEQ ID NO: 8. In another example, the TLR-7 is TLR-7 leucine-rich repeat (LRR) A.

In one example, the lipid nanoparticle additionally comprises a PEG-lipid, a structural lipid and/or a neutral lipid. For example, the lipid nanoparticle additionally comprises a PEG-lipid, a structural lipid and a neutral lipid. In another example, the lipid nanoparticle additionally comprises a PEG-lipid, a structural lipid or a neutral lipid. In one example, the lipid nanoparticle additionally comprises a PEG-lipid, a structural lipid, an ionisable lipid and/or a neutral lipid. For example, the lipid nanoparticle additionally comprises a PEG-lipid, a structural lipid, an ionisable lipid and a neutral lipid. In another example, the lipid nanoparticle additionally comprises a PEG-lipid, a structural lipid, an ionisable lipid or a neutral lipid.

In one example, the lipid nanoparticle additionally comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the lipid nanoparticle additionally comprises a structural lipid. For example, the structural lipid is selected from the group consisting of cholesterol, campesterol and combinations thereof.

In one example, the lipid nanoparticle additionally comprises a neutral lipid. For example, the neutral lipid is selected from the group consisting of DSPC, DOPE, DLPC, DMPC, DOPC, DPPC and combinations thereof.

In one example, the lipid nanoparticle additionally comprises an ionisable lipid.

In one example, the ionisable lipid is selected from the group consisting of: 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 1,2-dioleoyl-3-trimethylammonium propane (DOTAP), 1,2-distearyloxy-N,N-dimethyl-3-aminopropane (DSDMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N, N-dimethyl-3-[(9Z,12Z)-octadeca-9,12-dien-1-yloxy]propan-1-amine (Octyl-CLinDMA (2R)), (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino) butnoate (LKY750), 8-[(2-hydroxyethyl) [6-oxo-6-(undecyloxy) hexyl]amino]-octanoic acid, 1-octylnonyl ester (also referred to as heptadecan-9-yl 8-[2-hydroxyethyl-(6-oxo-6-undecoxyhexyl)amino]octanoate) (SM-102), 2-hexyl-decanoic acid, 1,1′-[[(4-hydroxybutyl)imino]di-6,1-hexanediyl]ester (also referred to as ((4-hydroxybutyl) azanediyl)bis(hexane-6,1-diyl)bis(2-hexyldecanoate)) (ALC-0315), 4-(dimethylamino)-butanoic acid, (10Z,13Z)-1-(9Z,12Z)-9,12-octadecadien-1-yl-10,13-nonadecadien-1-yl ester (DLin-MC3-DMA or MC3), ((4-hydroxybutyl) azanediyl)bis(hexane-6,1-diyl)bis(2-hexyldecanoate)), and 8-[(2-hydroxyethyl) [6-oxo-6-(undecyloxy) hexyl]amino]-octanoic acid, 1-octylnonyl ester and combinations thereof.

In one example, the lipid nanoparticle does not comprise an ionisable lipid.

In one example, the lipid nanoparticle does not comprise a cationic lipid.

In one example, the lipid nanoparticles have a mean particle size of between about 80 nm and 200 nm. For example, the lipid nanoparticles have a mean particle size of between about 100 nm and 200 nm. In one example, the lipid nanoparticles have a mean particle size of between about 100 nm and 190 nm, or about 100 nm and 180 nm, or about 110 nm and 180 nm, or about 110 nm and 150 nm, or about 110 nm and 140 nm, or about 110 nm and 130 nm. For example, the lipid nanoparticles have a mean particle size of about 125 nm. In one example, the lipid nanoparticles have a mean particle size of between about 150 and 200 nm. In one example, the lipid nanoparticles have a mean particle size of between about 160 and 200 nm. For example, the lipid nanoparticles has a mean particle size of about 160 nm, or about 165 nm, or about 170 nm, or about 175 nm, or about 180 nm, or about 185 nm, or about 190 nm, or about 200 nm. In one example, the mean particle size is determined by measuring the Z-average diameter of the lipid nanoparticles.

In one example, the lipid nanoparticles have a nitrogen to phosphate ratio of between about 2 to about 10. For example, the lipid nanoparticles have a nitrogen to phosphate ratio of about 2, or about 2.5, or about 3, or about 3.5, or about 4, or about 4.5, or about 5, or about 5.5, or about 6, or about 6.5, or about 7, or about 7.5, or about 8, or about 8.5, or about 9, or about 9.5, or about 10. In one example, the lipid nanoparticles have a nitrogen to phosphate ratio of about 3. In another example, the lipid nanoparticles have a nitrogen to phosphate ratio of about 4.5. In a further example, the lipid nanoparticles have a nitrogen to phosphate ratio of about 6.

In one example, at least 50% of the RNA is encapsulated within the lipid nanoparticles. For example, at least 50%, or at least 55%, or at least 60%, or at least 65%, or at least 70%, or at least 75%, or at least 80%, or at least 85%, or at least 90%, or at least 95% of the RNA is encapsulated within the lipid nanoparticles. In one example, at least 80% of the RNA is encapsulated. In another example, at least 85% of the RNA is encapsulated. It will be apparent to the skilled person that encapsulation efficiency (or percent encapsulation) may be determined by measuring the escape or the activity of the pharmaceutical composition or mRNA of the disclosure using fluorescence (e.g., using RiboGreen) and/or electron micrograph.

In one example, the RNA is selected from the group consisting of messenger RNA (mRNA), small-interfering RNA (siRNA), microRNA (miRNA) and antisense RNA.

In one example, the RNA is mRNA. For example, the mRNA is self-replicating mRNA (sa-mRNA) or conventional mRNA (cRNA). In one example, the mRNA is sa-mRNA. In another example, the mRNA is cRNA.

In one example, the RNA is siRNA.

In one example, the RNA is miRNA.

In one example, the RNA is antisense RNA.

The present disclosure also provides an immunogenic composition comprising the lipid nanoparticle of the present disclosure. For example, the composition of the present disclosure, when administered, is capable of inducing an immune response in the subject. For example, administration of the composition induces a humoral and/or a cell-mediated immune response. In one example, the composition induces a humoral immune response in the subject. For example, the humoral immune response is an antibody-mediated immune response. In another example, the composition induces a cell-mediated immune response. For example, the cell-mediated immune response includes activation of antigen-specific cytotoxic T cells.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

The present disclosure also provides the immunogenic composition or the pharmaceutical composition of the disclosure for use in therapy. For example, the immunogenic composition or the pharmaceutical composition of the disclosure is suitable for use as a vaccine.

In one example, the immunogenic composition or the pharmaceutical composition of the disclosure is supplied in a vial. In another example, the immunogenic composition or the pharmaceutical composition of the disclosure is supplied in a syringe.

In one example, the immunogenic composition or the pharmaceutical composition of the disclosure is stable for a period of at least 60 days at 4° C. In another example, the immunogenic composition or the pharmaceutical composition of the disclosure is stable for a period of at least 90 days at 4° C.

Additional embodiments of the disclosure:

1. A lipid nanoparticle for delivery of RNA, the lipid nanoparticle comprising therein a RNA-binding protein or peptide bound to the RNA.

2. The lipid nanoparticle of 1, wherein the RNA-binding protein or peptide is a lipidated RNA-binding protein or peptide.

3. A lipid nanoparticle for delivery of RNA, the lipid nanoparticle comprising therein a lipidated RNA-binding protein or peptide bound to the RNA.

4. The lipid nanoparticle of 2 or 3, wherein the RNA-binding protein or peptide is lipidated prior to binding the RNA.

5 The lipid nanoparticle of any one of 2 to 4, wherein the RNA-binding protein or peptide is lipidated with a lipid moiety selected from the group consisting of a fatty acid, an isoprenoid and combinations thereof.

6. The lipid nanoparticle of 5, wherein the fatty acid is a triglyceride, a phospholipid or a cholesteryl ester.

7. The lipid nanoparticle of any one of 2 to 6, wherein the RNA-binding protein or peptide is lipidated on a nucleophilic side chain, at the N-terminal end and/or at the C-terminal end.

8 The lipid nanoparticle of 7, wherein the nucleophilic side chain is a cysteine, a serine, a threonine, a tyrosine and/or a lysine amino acid residue.

9. The lipid nanoparticle of any one of 2 to 8, wherein the RNA-binding protein or peptide is lipidated by palmitoylation, myristoylation, fatty-acylation, esterification, prenylation, or combinations thereof.

10. The lipid nanoparticle of 9, wherein the RNA-binding protein or peptide is lipidated by N-terminal cysteine palmitoylation, N-terminal glycine myristoylation, lysine N-acylation, C-terminal cholesterol esterification, cysteine prenylation, serine O-acylation or combinations thereof.

11. The lipid nanoparticle of 9 or 10, wherein the prenylation is farnesylation or geranylgeranylation.

12. The lipid nanoparticle of any one of 5 to 11, wherein the lipid moiety is linked to the RNA-binding protein or peptide by a thioether bond, an ester bond, a thioester bond and/or an amide bond.

13. The lipid nanoparticle of any one of 2 to 12, wherein the RNA-binding protein or peptide is lipidated using chemical or enzymatic lipidation.

14. The lipid nanoparticle of 13, wherein the RNA-binding protein or peptide is lipidated using chemical lipidation selected from the group consisting of chemical ligation, click chemistry, expressed protein ligation and combinations thereof.

15. The lipid nanoparticle of 13, wherein the RNA-binding protein or peptide is lipidated using enzymatic lipidation selected from the group consisting of Sortase-A mediated lipidation, transglutaminase mediated lipidation and combinations thereof.

16. The lipid nanoparticle of 15, wherein the enzymatic lipidation is performed in vivo or in vitro.

17. The lipid nanoparticle of any one of 1 to 16, wherein the RNA-binding protein or peptide encapsulates the RNA.

18. The lipid nanoparticle of any one of 1 to 17, wherein the RNA-binding protein or peptide binds directly to the RNA.

19. The lipid nanoparticle of any one of 1 to 18, wherein the RNA-binding protein or peptide:
a) reduces toxicity of the lipid nanoparticle, and/or
b) stabilizes the RNA, and/or
c) protects the RNA from degradation, and/or
d) facilitates nucleation of the lipid nanoparticle, and/or
e) inhibits induction of signalling by one or more Toll-like Receptors.

20. The lipid nanoparticle of any one of 1 to 19, wherein the RNA-binding protein or peptide is modified to remove nuclear localisation signal(s) and/or introduce nuclear export signal(s).

21. The lipid nanoparticle of any one of 1 to 20, wherein the RNA-binding protein or peptide is a viral or non-viral RNA-binding protein or peptide.

22. The lipid nanoparticle of 21, wherein the viral RNA-binding protein is from a class III, class IV, class V and/or class VI virus.

23. The lipid nanoparticle of 22, wherein the viral RNA binding protein or peptide is a respiratory virus selected from the group consisting of an influenza virus, a respiratory syncytial virus, a parainfluenza virus, a metapneumovirus, a rhinovirus, a coronavirus, an adenovirus and a bocavirus.

24. The lipid nanoparticle of 23, wherein the viral RNA-binding protein or peptide is a nucleoprotein, a non-structural protein, a matrix protein and/or a nucleocapsid protein.

25. The lipid nanoparticle of any one of 21 to 24, wherein the viral RNA-binding protein or peptide is a non-structural (NS) protein from an influenza B virus.

26. The lipid nanoparticle of any one of 21 to 25, wherein the viral RNA-binding protein or peptide comprises a sequence set forth in any one of SEQ ID NOs: 9 to 11.

27. The lipid nanoparticle of 21, wherein the non-viral RNA binding protein or peptide is derived from a cellular protein associated with cell growth, cell signalling and/or anti-viral pathways.

28. The lipid nanoparticle of 27, wherein the cellular protein is selected from the group consisting of a TAR RNA-binding protein (TRBP), a protein kinase R (PKR) RNA-binding protein, a Toll-like Receptor 3 (TLR-3) binding protein, a TLR-7 binding protein and combinations thereof.

29. The lipid nanoparticle of 27 or 28, wherein the cellular protein comprises a sequence set forth in any one of SEQ ID NOs: 1 to 8.

30. The lipid nanoparticle of any one of 1 to 29, wherein the lipid nanoparticle additionally comprises a PEG-lipid, a structural lipid and/or a neutral lipid.

31. The lipid nanoparticle of any one of 1 to 30, wherein the lipid nanoparticle does not comprise a cationic lipid.

32. The lipid nanoparticle of any one of 1 to 31, wherein the RNA is selected from the group consisting of messenger RNA (mRNA), small-interfering RNA (siRNA), microRNA (miRNA) and antisense RNA.

33. The lipid nanoparticle of 29, wherein the mRNA is self-replicating mRNA (sa-mRNA) or a conventional RNA (cRNA).

34. An immunogenic composition comprising the lipid nanoparticle of any one of 1 to 33.

35. A pharmaceutical composition comprising the lipid nanoparticle of any one of 1 to 33 or the immunogenic composition of 34 and a pharmaceutically acceptable carrier.

36. The lipid nanoparticle of any one of 1 to 33, immunogenic composition of 34 or the pharmaceutical composition of 35 for use in therapy.

### Key to Sequence Listing

SEQ ID NO: 1 Amino acid sequence of full length TAR RNA-binding protein domain 2
SEQ ID NO: 2 Amino acid sequence of truncated TAR RNA-binding protein domain 2
SEQ ID NO: 3 Amino acid sequence of protein kinase R RNA-binding motif 2
SEQ ID NO: 4 Amino acid sequence of truncated protein kinase R RNA-binding motif 2 (#1)
SEQ ID NO: 5 Amino acid sequence of truncated protein kinase R RNA-binding motif 2 (#2)
SEQ ID NO: 6 Amino acid sequence of Toll-like receptor 3 dsRNA-binding domain 1 (leucine-rich repeats 1-3)
SEQ ID NO: 7 Amino acid sequence of Toll-like receptor 3 dsRNA-binding domain 1 (leucine-rich repeats 17-18)
SEQ ID NO: 8 Amino acid sequence of Toll-like receptor 7 RNA-binding site (leucine-rich repeats 14-15)
SEQ ID NO: 9 Amino acid sequence of influenza B NS1 RNA-binding domain
SEQ ID NO: 10 Amino acid sequence of truncated influenza B NS1 RNA-binding domain
SEQ ID NO: 11 Amino acid sequence of modified influenza B NS1 RNA-binding domain

### BRIEF DESCRIPTION OF THE DRAWINGS

FIG. 1 is a graphical representation of a rabbit reticulocyte lysate assay showing the amount of RNA as measured by the amount of luciferase produced as assessed by measuring luminescence in relative light units (RLU) in nanoluciferase RNA (nLuc RNA) alone or in combination with influenza virus RNA-free nucleoprotein (NP: nLuc RNA).

FIG. 2 is a graphical representation of the amount of RNA in (A) NP: nLuc 10 RNA and (B) nLuc RNA alone samples following treatment with or without thermolabile proteinase K (PK) and/or RNase.

FIG. 3 is a series of graphical representations showing the stability of nLuc RNA in samples of nLuc RNA alone and NP: nLuc RNA at (A) 4° C. (B) 24° C. and (C) 37° C. following incubation for up to 96 hours.

FIG. 4 is a series of graphical representations showing the level of (A-B) TLR3 and (C-D) TLR8 induction in samples of nLuc RNA alone and NP: nLuc RNA at 2 hours and 4 hours post incubation.

FIG. 5 is a series of graphical representations showing (A) protection of RNA degradation by SARS-CoV-2 nucleocapsid as measured by the amount of luciferase produced as assessed by measuring luminescence in RLU in nanoluciferase RNA (RNA) alone or in combination with COVID RNA-free nucleocapsid (RNA+NP (SCov2)). (B) Dye exclusion assay of nLuc RNA with and without COVID RNA-free nucleocapsid.

FIG. 6 is a graphical representations showing the level of protection afforded by RNA binding peptides reacted with RNA compared to free RNA. The dashed lines represent the level of protection with no peptide present (LHS) equivalent to free RNA and 100% protection.

FIG. 7 is a series of graphical representations showing the expression of nLuc RNA in samples NP: nLuc RNA (A) with no RNase inhibitor (B) with an RNase inhibitor (C) with no RNase inhibitor and (D) with an RNase inhibitor.

FIG. 8 is a graphical representation showing nLuc expression (measured as relative light units; RLU) in Hela cells transfected with NP: nLuc RNA and nLuc RNA.

FIG. 9 is a graphical representation showing nLuc RNA expression in the spleen and liver.

FIG. 10 is a graphical representation showing stability of LNPs formulated with nLuc mRNA or NP-nLuc mRNA over time at 4° C. following incubation for up to 90 days.

FIG. 11 is a series of graphical representations showing the biodistribution of LNPs formulated with nLuc mRNA and LNPs formulated with nLuc mRNA and nucleoprotein (NP) at a MC3: PEG ratio of (A) 0:1.5 (B) 5:1.5 (C) 10:1.5 (D) 15:15 (E) 20:1.5 and (F) 50:1.5.

FIG. 12 is a graphical representation of LNPs formulated with mRNA or mRNA NuP showing (A) LNP sizes, (B) encapsulation efficiency and (C) biodistribution with altering the nitrogen to phosphate ratio.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

As used herein, the term “lipid nanoparticle” or “LNP” shall be understood to refer to lipid-based particles having at least one dimension on the order of nanometers (e.g., 1-1,000 nm) and which comprises a compound of any formulae described herein. In embodiments, LNPs are formulated in a composition for delivery of a polynucleotide to a desired target such as a cell, tissue, organ, tumor, and the like. For example, the lipid nanoparticle or LNP may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

The term “lipidated” or “lipidation” as used herein refers to the process of covalently modifying a protein (i.e., a RNA-binding protein or peptide) with one or more lipids.

As used herein, the term “RNA-binding protein or peptide” or “RBP’ shall be understood to refer to proteins and peptides that bind to double or single stranded RNA and participate in forming ribonucleoprotein complexes.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “peptide” as used herein is intended to include compounds composed of amino acid residues linked by amide bonds. A peptide may be natural or unnatural, ribosome encoded or synthetically derived. Typically, a peptide will consist of between 2 and 200 amino acids. For example, the peptide may have a length in the range of 10 to amino acids or 10 to 30 amino acids or 10 to 40 amino acids or 10 to 50 amino acids 20 or 10 to 60 amino acids or 10 to 70 amino acids or 10 to 80 amino acids or 10 to 90 amino acids or 10 to 100 amino acids, including any length within said range(s).

As used herein, the term “recombinant” shall be understood to mean the product of artificial genetic recombination.

As used herein, the term “self-replicating RNA” refers to a construct based on an RNA virus that has been engineered to allow expression of heterologous RNA and proteins. Self-replicating RNA (e.g., in the form of naked RNA) can amplify in host cells leading to expression of the desired gene product in the host cell.

As used herein, the term “conventional RNA” or “CRNA” or “non-amplifying RNA” refers to a construct that allows expression of heterologous RNA and proteins but the RNA that cannot amplify in host cells.

As used herein, the term “subject” shall be taken to mean any animal including humans, for example a mammal. Exemplary subjects include but are not limited to humans and non-human primates. For example, the subject is a human.

### Lipid Nanoparticles

The present disclosure provides a lipid nanoparticle for delivery of RNA, wherein the lipid nanoparticle comprises a nucleic acid-binding protein or peptide bound to the RNA. For example, the lipid nanoparticle comprises a lipidated nucleic acid-binding protein or peptide bound to the RNA.

The present disclosure provides a lipid nanoparticle for delivery of RNA, wherein the lipid nanoparticle comprises a lipidated RNA-binding protein or peptide bound to the RNA.

Nucleic acid-binding proteins or peptides

The present disclosure provides a lipid nanoparticle comprising a nucleic acid-binding protein or peptide. For example, the present disclosure provides a lipid nanoparticle comprising a lipidated nucleic acid-binding protein or peptide.

In one example, the nucleic acid-binding protein is a RNA-binding protein or peptide. In another example, the nucleic acid-binding protein is a RNA- and DNA-binding protein or peptide.

RNA-binding proteins or peptides

The present disclosure provides a lipid nanoparticle comprising a RNA-binding protein or peptide. For example, the present disclosure provides a lipid nanoparticle comprising a lipidated RNA-binding protein or peptide.

RNA-binding proteins regulate numerous aspects of co- and post-transcription gene expression including, for example, RNA splicing, RNA editing, polyadenylation, export, mRNA stabilization, mRNA localization and translation. RNA-binding proteins or peptide bind to double or single-stranded RNA and participate in the formation of ribonucleoprotein complexes. The skilled person will understand that RNA-binding proteins or peptides can be viral or non-viral proteins or peptides.

Non-viral RNA-binding proteins or peptides

In one example, the RNA-binding protein is a non-viral protein or peptide derived from cellular proteins. For example, RNA-binding proteins or peptides are derived from cellular proteins associated with cell growth, cell signalling and/or anti-viral pathways.

Non-viral RNA-binding proteins or peptides contain numerous structural motifs or RNA-binding domains that facilitate RNA binding including, for example, a RNA recognition motif (RRM), a K-homology (KH) domain (type I and type II), a RGG (Arg-Gly-Gly) box, a Sm domain; DEAD/DEAH box, a CCCH-type zinc finger (ZnF), a double stranded RNA-binding motif (dsRBD), a cold-shock domain; Pumilio/FBF (PUF or Pum-HD) domain, and a Piwi/Argonaute/Zwille (PAZ) domain.

In one example, the RNA-binding protein or peptide comprises a RNA-binding domain selected from the group consisting of a RNA recognition motif, a K-homology domain (type I or type II) and a CCCH-type zinc finger.

In one example, the RNA-binding protein or peptide comprises a RNA recognition motif. For example, the RNA-binding protein or peptide comprising a RNA recognition motif is selected from the group consisting of A2BP1, ACF, BOLL, BRUNOL4, BRUNOL5, BRUNOL6, CCBL2, CGI-96, CIRBP, CNOT4, CPEB2, CPEB3, CPEB4, CPSF7, CSTF2, CSTF2T, CUGBP1, CUGBP2, D10S102, DAZ1, DAZ2, DAZ3, DAZ4, DAZAPI, DAZL, DNAJC17, DND1, EIF3S4, EIF3S9, EIF4B, EIF4H, ELAVL1, ELAVL2, ELAVL3, ELAVL4, ENOX1, ENOX2, EWSRI, FUS, FUSIP1, G3BP, G3BP1, G3BP2, GRSF1, HNRNPL, HNRPA0, HNRPA1, HNRPA2B1, HNRPA3, HNRPAB, HNRPC, HNRPCL1, HNRPD, HNRPDL, HNRPF, HNRPH1, HNRPH2, HNRPH3, HNRPL, HNRPLL, HNRPM, HNRPR, HRNBP1, HSU53209, HTATSF1, IGF2BP1, IGF2BP2, IGF2BP3, LARP7, MKI67IP, MSI1, MSI2, MSSP-2, MTHFSD, MYEF2, NCBP2, NCL, NOL8, NONO, P14, PABPCI, PABPCIL, PABPC3, PABPC4, PABPC5, PABPNI, PKR, POLDIP3, PPARGC1, PPARGCIA, PPARGCIB, PPIE, PPIL4, PPRC1, PSPC1, PTBP1, PTBP2, PUF60, RALY, RALYL, RAVER1, RAVER2, RBM10, RBM11, RBM12, RBM12B, RBM14, RBM15, RBM15B, RBM16, RBM17, RBM18, RBM19, RBM22, RBM23, RBM24, RBM25, RBM26, RBM27, RBM28, RBM3, RBM32B, RBM33, RBM34, RBM35A, RBM35B, RBM38, RBM39, RBM4, RBM41, RBM42, RBM44, RBM45, RBM46, RBM47, RBM4B, RBM5, RBM7, RBM8A, RBM9, RBMS1, RBMS2, RBMS3, RBMX, RBMX2, RBMXL2, RBMYIAI, RBMYIB, RBMYIE, RBMYIF, RBMY2FP, RBPMS, RBPMS2, RDBP, RNPC3, RNPC4, RNPSI, RODI, SAFB, SAFB2, SART3, SERBPI, SETDIA, SF3B14, SF3B4, SFPQ, SFRS1, SFRS10, SFRS11, SFRS12, SFRS15, SFRS2, SFRS2B, SFRS3, SFRS4, SFRS5, SFRS6, SFRS7, SFRS9, SLIRP, SLTM, SNRP70, SNRPA, SNRPB2, SPEN, SR140, SRRP35, SSB, SYNCRIP, TAF15, TRBP, THOC4, TIAI, TIALI, TNRC4, TNRC6C, TRA2A, TRSPAPI, TUTI, UISNRNPBP, U2AF1, U2AF2, UHMK1, ZCRB1, ZNF638, ZRSR1 and ZRSR2.

In one example, the RNA-binding protein or peptide comprises a K-homology domain. For example, the K-homology domain is a type I domain. In another example, the K-homology domain is a type II domain. In one example, the RNA-binding protein or peptide comprising a K-homology domain is selected from the group consisting of AKAP1, ANKHD1, ANKRD17, ASCC1, BICCI, DDX43, DDX53, DPPA5, FMRI, FUBP1, FUBP3, FXR1, FXR2, GLD1, HDLBP, HNRPK, IGF2BP1, IGF2BP2, IGF2BP3, KHDRBS1, KHDRBS2, KHDRBS3, KHSRP, KRR1, MEX3A, MEX3B, MEX3C, MEX3D, NOVA1, NOVA2, PCBP1, PCBP2, PCBP3, PCBP4, PNO1, PNPT1, QKI, SF1, and TDRKH.

In one example, the RNA-binding domain comprises a CCCH-type zinc finger domain.

Exemplary non-viral RNA-binding protein or peptide will be apparent to the skilled person and include, for example, TAR RNA-binding protein (TRBP), protein kinase R (PKR), Toll-like receptor 3 (TLR-3) and Toll-like receptor 7 (TLR).

Viral RNA-binding proteins

In one example, the RNA-binding protein or peptide is a viral RNA-binding protein or peptide. For example, the RNA-binding protein is a nucleoprotein, a matrix protein, a nucleocapsid protein and/or a non-structural from a RNA virus.

It will be apparent to the skilled person that viruses are classified according to the Baltimore classification system, as shown in Table 1, which is largely based on the transcription of the viral genome.

TABLE 1
Baltimore classification of viruses

| Class | Genome |
|---|---|
| I | double-stranded DNA viruses |
| II | single-stranded DNA viruses |
| III | double-stranded RNA viruses |
| IV | positive sense single-stranded RNA viruses |
| V | negative sense single-stranded RNA viruses |
| VI | single-stranded RNA viruses with a DNA |
| | intermediate in their life cycle |
| VII | double-stranded DNA viruses with a RNA |
| | intermediate in their life cycle |

In one example, the RNA-binding protein or peptide is from a RNA virus. For example, the RNA-binding protein or peptide is from a class III, a class IV, a class V and/or a class VI virus.

In one example, the RNA virus is a class III virus (i.e., a double-stranded RNA virus). Class III viruses include, for example, all viruses of the phylum Duplornaviricota and all viruses of class Duplopiviricetes (of phylum Pisuviricota). Exemplary class III viruses include, but are not limited to, Reoviruses (e.g., Orthoreo virus, a Rotavirus, an Orbivirus, or a Coltivirus).

In one example, the RNA virus is a class IV virus (i.e., positive sense single-stranded RNA virus). Class IV viruses include, for example, viruses of the phylum Lenarviricota, Pisuviricota (except of the class Duplopidiviricetes) and Kitrinoviricota. Exemplary class IV viruses include, but are not limited to, Togaviruses (e.g., Rubivirus, an Alphavirus, or an Arterivirus), Flaviviruses (e.g., Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus), Picornaviruses (e.g., Enteroviruses, Rhinoviruses, Heparnavirus, Parechovirus, Cardioviruses and Aphthoviruses), Enteroviruseses (e.g., Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71), Pestiviruses (e.g., Bovine viral diarrhea (BVDV), Classical swine fever (CSFV) or Border disease (BDV)), Caliciviridae (e.g., Norwalk virus, and Norwalk-like Viruses (e.g., Hawaii Virus and Snow Mountain Virus), Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-COV), Avian infectious bronchitis (IBV), Mouse hepatitis virus (MHV), and Porcine transmissible gastroenteritis virus (TGEV)) and Hepatitis E virus (HEV).

In one example, the RNA virus is a class V virus (i.e., negative sense single-stranded RNA virus). Class V viruses include, for example, viruses of the phylum Negarnaviricota. Exemplary class V viruses include, but are not limited to, Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxovirus types 1-4 (PIV), Mumps, Sendai viruses, Simian virus 5), Nipahvirus, Henipavirus, Newcastle disease virus, Morbilliviruses (e.g., Measles), Bunyaviruses (e.g., California encephalitis virus), Phlebovirus (e.g., Rift Valley Fever virus), Nairovirus (e.g., Crimean-Congo hemorrhagic fever virus), Rhabdoviruses (e.g., Lyssavirus (Rabies virus) and Vesiculovirus (VSV)), Delta hepatitis virus (HDV) and Arenaviruses.

In one example, the class V RNA virus is an influenza virus. For example, an influenza A virus. In another example, an influenza B virus.

In one example, the RNA virus is a class VI virus (i.e., single-stranded RNA viruses with a DNA intermediate in their life cycle). Class VI viruses include, for example, viruses of the class Revtraviricetes (of phylum Aterviricota, excluding (aulimoviridae). Exemplary class VI viruses include, but are not limited to, Heparnaviruses (e.g., Hepatitis A virus (HAV)), Hepadnaviruses (e.g., Hepatitis B virus, Hepatitis C virus) and Retroviruses (e.g., Oncovirus, a Lentivirus or a Spumavirus). In one example, the RNA virus is a hepadnavirus. For example, the hepadnavirus is hepatitis B virus (HBV).

### Linkers

In one example, the RNA-binding protein or peptide comprises a first RNA-binding protein or peptide and a second RNA-binding protein or peptide linked via a linker. For example, the linker is a linker peptide.

In one example, the linker is a flexible linker.

A “flexible” linker is an amino acid sequence which does not have a fixed structure (secondary or tertiary structure) in solution. Such a flexible linker is therefore free to adopt a variety of conformations. Flexible linkers suitable for use in the present disclosure are known in the art. An example of a flexible linker for use in the present invention is the linker sequence SGGGGS/GGGGS/GGGGS or (Gly₄Ser)₃. Another example of a flexible linker is an alanine linker (e.g., Alaₙ).

The linker may comprise any amino acid sequence that does not substantially hinder interaction of the RNA-binding protein or peptide with the RNA. Preferred amino acid residues for flexible linker sequences include, but are not limited to, glycine, alanine, serine, threonine proline, lysine, arginine, glutamine and glutamic acid.

The linker sequences between the RNA-binding protein or peptide preferably comprise five or more amino acid residues. The flexible linker sequences according to the present disclosure consist of 5 or more residues, preferably, 5, 6, 7, 8, 9, 10, 11, 12, 13, 14, 15, 16, 17, 18, 19 or 20 or more residues. In a highly preferred embodiment of the invention, the flexible linker sequences consist of 5, 7, 10 or 16 residues.

In one example, the linker is a rigid linker. A “rigid linker” (including a “semi-rigid linker”) refers to a linker having limited flexibility. For example, the relatively rigid linker comprises the sequence (EAAAK)ₙ, where n is between 1 and 3. The value of n can be between 1 and about 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 70, or less than about 60, or less than about 50, or less than about 40, or less than about 30, or less than about 20, or less than about 10. A rigid linker need not completely lack flexibility.

### Lipidation of the Binding Protein

The present disclosure provides a lipid nanoparticle comprising a lipidated nucleic acid-binding protein or peptide.

The present disclosure provides a lipid nanoparticle comprising a lipidated RNA-binding protein or peptide.

It will be apparent to the skilled person that protein or peptide lipidation is the covalent attachment of a lipid moiety to the protein or peptide (i.e., RNA-binding protein or peptide).

### Lipids

Lipid moieties suitable for use in the present disclosure will be apparent to the skilled person and include, for example, a fatty acid, an isoprenoid and combinations thereof. In one example, the lipid moiety is selected from the group consisting of an isoprenoid, a triglyceride, a phospholipid, a cholesteryl ester and combinations thereof.

### Isoprenoids

Isoprenoids, also known as terpenoids or prenol lipids, are branched lipids and are a class of organic compounds composed of two or more units of hydrocarbons, with each unit consisting of five carbon atoms arranged in a specific pattern. These five-carbon units are termed isoprene and are synthesized from a common intermediate known as mevalonic acid, which is itself synthesized from acetyl-CoA. Isoprenoids can have one or more functional chemical groups attached to their carbon backbone, such as hydroxyls and carbonyls, which make up the diversity of isoprenoids. Isoprenoids can be classified as monoterpenes (C₁₀H₁₆) sesquiterpenes (C₁₅H₂₄), diterpenes (C₂₀H₃₂), triterpenes (C₃₀H₄₈), tetraterpenes (C₄₀H₆₄) or other polyterpenes (C₅H₈) n.

Isoprenoids suitable for use in the present disclosure will be apparent to the skilled person and/or are described herein.

In one example, the isoprenoid is a monoterpene. Exemplary monoterpenes include citronellol, citronellal, citral, geraniol, methol, pseudoionone and beta-ionone.

In one example, the isoprenoid is a sesquiterpenes. Exemplary sesquiterpenes include cadalene, eudalene, cadinene and beta-selinene.

In one example, the isoprenoid is a diterpene. Exemplary diterpenes include phytol and abietic acid.

In one example, the isoprenoid is a triterpene. Exemplary triterpenes include squalene and beta-amyrin.

In one example, the isoprenoid is a tetraterpene. Exemplary tetraterpenes include carotenoids (e.g., beta-carotene) and lycopene.

### Fatty Acids

Fatty acids are lipids that contain long-chain hydrocarbons terminated with a carboxylic acid functional group. Fatty acids may be saturated or unsaturated. In one example, the fatty acid comprises a carbon chain having from 6 to 22 carbons. Exemplary fatty acids include palmitic acid, myristic acid, oleic acid, alpha-linolenic acid and stearic acid.

Fatty acids rarely occur in the free form in nature and commonly exist as three main classes of esters: triglycerides, phospholipids and cholesteryl esters.

In one example, the fatty acid is a triglyceride. Triglycerides are tri-esters consisting of a glycerol bound to three fatty acid molecules via an ester bond. The three fatty acids may be the same or different. An exemplary triglyceride is tristearin.

In one example, the fatty acid is a phospholipid. Phospholipids are complex lipids that comprise a hydrophilic polar head group comprising one or more phosphate groups, and a hydrophobic tail comprising two fatty acyl chains. The polar head group is joined to the hydrophobic moiety by a phosphodiester linkage via a glycerol (i.e., phosphoglycerides) or sphingosine molecule (i.e., phosphosphingo lipids). Phospholipids may be saturated or unsaturated. Exemplary phosphoglycerides include phosphatidic acid (phosphatidate), phosphatidylethanolamine (cephaline), phosphatidylcholine (lecithin), phosphatidylserine, phosphoinositides (e.g., phosphatidylinositol (PI), phosphatidylinositol phosphate (PIP), phosphatidylinositol bisphosphate (PIP2) and phosphatidylinositol trisphosphate (PIP3)), phosphatidylglycerol and cardiolipin. Exemplay phosphosphingo lipids include ceramide phosphorylcholine (sphingomyelin), ceramide phosphorylethanolamine (sphingomyelin), ceramind phosphoryllipid, galactocerebroside, glucocerebroside and lactosylceramide.

In one example, the fatty acid is a cholesteryl ester. Cholesteryl esters are the esterification of cholesterol with long-chain fatty acids. Exemplary cholesteryl esters include cholesteryl oleate, cholesteryl benzoate and cholesteryl linoleate.

### Lipidation

Exemplary lipidation includes, palmitoylation, myristoylation, fatty-acylation, esterification, prenylation, or combinations thereof.

### Palmitoylation

In one example, the lipid moiety is attached to the RNA-binding protein or peptide by palmitoylation.

In one example, the palmitoylation is cysteine palmitoylation (also known as S-palmitoylation). The skilled person will understand that cysteine palmitoylation is the addition of a 16-carbon palmitoyl group on protein cysteine residues. In one example, the palmitoyl group is added via a thioester bond. In another example, the palmitoyl group is added via an amide bond.

### Myristoylation

In one example, the lipid moiety is attached to the RNA-binding protein or peptide by myristoylation.

In one example, the myristoylation is N-glycine myristoylation. The skilled person will recognise that N-glycine myristoylation refers to the co- or post-translational attachment of a saturated 14-carbon fatty acyl group, myristoyl, to the N-terminal glycine of proteins via an amide bond.

In one example, the myristoylation is lysine myristoylation.

### Fatty-acylation

In one example, the lipid moiety is attached to the RNA-binding protein or peptide by fatty-acylation.

The skilled person will recognise that fatty-acylation involves the covalent attachment of an acyl group to a protein.

In one example, the fatty-acylation is lysine N-acylation. The skilled person will understand that lysine N-acylation refers to the transfer of the acetyl moiety from acetyl-CoA to the epsilon (ε)-amino group of a lysine residue on a protein.

### Esterification

In one example, the lipid moiety is attached to the RNA-binding protein or peptide by esterification.

In one example, the esterification is C-terminal sterol esterification, for example C-terminal cholesterol esterification. The skilled person will understand that C-terminal cholesterol esterification is the replacement of at least one hydroxyl (—OH) group with an alkoxy (—O-alkyl) group.

### Prenylation

In one example, the lipid moiety is attached to the RNA-binding protein or peptide by prenylation.

In one example, the prenylation is cysteine prenylation. The skilled person will understand that cysteine prenylation is the addition of multiple isoprene units to cysteine residues near the C-terminal end of the protein.

In one example, the prenylation is farnesylation (i.e., the addition of three isoprene units), or the prenylation is geranylgeranylation (i.e., the addition of four isoprene units).

In one example, the linkage between farnesyl or geranylgeranyl groups and cysteine residues is a thioether bond. In another example, the linkage is an ester bond. In a further example, the linkage is a thioester bond.

### Methods of Lipidation

Lipid modifications typically occur on the nucleophilic side chains of proteins or peptide (e.g., cysteine, serine and lysine), at the N-terminal end and/or at the C-terminal end of proteins or peptides.

Various methods of lipidation will be apparent to the skilled person and/or are described herein. Suitable methods can include chemical or enzymatic lipidation.

### Chemical Lipidation

In one example, the lipid moiety is attached to the RNA-binding protein or peptide using chemical ligation. The lipid moiety can comprise an amine, carboxylic acid, hydrazide, or maleimide group and the lipid moiety may be chemically coupled to the RNA-binding protein or peptide via the primary amine group of a lysine or the thiol group of a cysteine. In one example, the lipid moiety comprises a maleimide group and the lipid moiety is attached to the RNA-binding protein or peptide via the formation of a thioether bond with a sulphydryl group in the RNA binding protein or peptide. In one example, the lipid moiety comprises a carboxylic acid and the carboxylic acid is activated by 1-ethyl-3-(3-dimethylaminopropyl) carbodiimide (EDC) and N-hydroxysulfosuccinimide (Sulfo-NHS). The activated acyl amino ester or sulfo-NHS ester subsequently reacts with a primary amine of a lysine residue in the RNA binding protein or peptide forming an amide bond.

In one example, the lipid moiety comprises a maleimide group. For example, the lipid moiety is a phospholipid capped with a maleimide group. In one example, the lipid moiety is a 1,2-Distearoyl-sn-Glycero-3-Phosphoethanolamine-maleimide (DSPE-maleimide; DSPE-Mal).

In one example, the lipid moiety is attached to the RNA-binding protein or peptide using various “click chemistry” strategies such as those disclosed in Kolb et al. (2001), WO 2003/101972 and Malkoch et al. (2005).

In one example, the lipid moiety is attached to the RNA-binding protein or peptide using expressed protein ligation. Expressed protein ligation comprises chemoselective ligation between a protein or peptide with a C-terminal thioester and a protein or peptide with an N-terminal cysteine in aqueous solution at physiological pH. In one example, the C-terminal thioester is inserted into the RNA binding protein or peptide by genetic manipulation and the lipid moiety is fused to a peptide having an N-terminal cysteine residue.

Other methods of chemical lipidation known to the skilled person may be used, such as those disclosed in Takahara & Kamiya (2020).

### Enzymatic Lipidation

In one example, the lipid moiety is attached to the RNA-binding protein or peptide using enzymatic lipidation. Enzymatic lipidation may be performed in vivo or in vitro. In some examples, the RNA-binding protein or peptide is genetically manipulated using techniques known to the skilled person to comprise a consensus sequence recognized by the lapidating enzyme.

In one example, the lipid moiety is attached to the RNA-binding protein or peptide using Sortase-A mediated lipidation. Sortase A (e.g., SrtA from Staphylococcus aureus) covalently attaches secreted proteins to a bacterial cell wall peptidoglycan in the presence of Ca²⁺ via a transpeptidation reaction. In this example, the RNA-binding protein or peptide is genetically manipulated to comprise an LPXTG motif (e.g., LPETG) at the C-terminus and the lipid moiety comprises a nucleophile and an oligo-glycine motif (e.g., triglycine, tetraglycine or pentaglycine). Upon addition of the sortase, the RNA binding-protein or peptide is covalently linked to the lipid through a peptide bond.

In one example, the lipid moiety is attached to the RNA-binding protein or peptide using transglutaminase mediated lipidation. Transglutaminase (e.g., Microbial transglutaminase: MTG) catalyzes a reaction between a glutamine residue and a lysine residue in a peptide or protein in the absence Ca²⁺ forming an irreversible cross-link. In one example, the RNA-binding protein or peptide is genetically manipulated to comprise the MTG lysine recognition sequence (e.g., MRHKGS), for example at the N- or C-terminus, and the lipid moiety comprises the MTG glutamine recognition sequence (e.g., LLQG). In one example, the RNA-binding protein or peptide is genetically manipulated to comprise the MTG glutamine recognition sequence (e.g., LLQG or LQ), for example at the N- or C-terminus, and the lipid moiety comprises MTG lysine recognition sequence (e.g., MRHKGS).

Other methods of enzymatic lipidation known to the skilled person may be used, such as those disclosed in Takahara & Kamiya (2020).

### Additional Lipids

In one example, the lipid nanoparticle additionally comprises a PEG-lipid, a sterol structural lipid and/or a neutral lipid. In one example, the lipid nanoparticle additionally comprises a PEG-lipid, a sterol structural lipid, an ionisable lipid and/or a neutral lipid. In one example, the lipid nanoparticle does not comprise a cationic lipid.

### PEG-lipids

In one example, the present disclosure provides a lipid nanoparticle comprising a PEGylated lipid.

It will be apparent to the skilled person that reference to a PEGylated lipid is a lipid that has been modified with polyethylene glycol. Exemplary PEGylated lipids include, but are not limited to, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid includes PEG-c-DMG, PEG-DMG, PEG-DLPE, PEG-DMPE, PEG-DPPC, a PEG-DSPE lipid and combinations thereof.

### Neutral Lipids

In one example, the present disclosure provides a lipid nanoparticle comprising a neutral lipid.

Suitable neutral or zwitterionic lipids for use in the present disclosure will be apparent to the skilled person and include, for example, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. The lipids can be saturated or unsaturated.

### Structural Lipids

In one example, the present disclosure provides a lipid nanoparticle comprising a structural lipid.

Exemplary structural lipids include, but are not limited to, cholesterol fecosterol, sitosterol, campesterol, stigmasterol, brassicasterol, ergosterol, tomatidine, tomatine, ursolic acid and alpha-tocopherol.

In one example, the structural lipid is a sterol. For example, the structural lipid is cholesterol. In another example, the structural lipid is campesterol.

### Ionisable Lipids

In one example, the present disclosure provides a lipid nanoparticle comprising an ionisable lipid.

Suitable ionisable lipids for use in the present disclosure will be apparent to the skilled person and include, for example, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 1,2-dioleoyl-3-trimethylammonium propane (DOTAP), 1,2-distearyloxy-N,N-dimethyl-3-aminopropane (DSDMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino) butnoate (LKY750), 8-[(2-hydroxyethyl) [6-oxo-6-(undecyloxy) hexyl]amino]-octanoic acid, 1-octylnonyl ester (also referred to as heptadecan-9-yl 8-[2-hydroxyethyl-(6-oxo-6-undecoxyhexyl)amino]octanoate) (SM-102), 2-hexyl-decanoic acid, 1,1′-[[(4-hydroxybutyl)imino]di-6,1-hexanediyl]ester (also referred to as ((4-hydroxybutyl) azanediyl)bis(hexane-6,1-diyl)bis(2-hexyldecanoate)) (ALC-0315), 4-(dimethylamino)-butanoic acid, (10Z,13Z)-1-(9Z,12Z)-9,12-octadecadien-1-yl-10,13-nonadecadien-1-yl ester (DLin-MC3-DMA or MC3), ((4-hydroxybutyl) azanediyl)bis(hexane-6,1-diyl)bis(2-hexyldecanoate)), and 8-[(2-hydroxyethyl) [6-oxo-6-(undecyloxy) hexyl]amino]-octanoic acid, 1-octylnonyl ester and combinations thereof.

### Pharmaceutically Acceptable Carrier

Suitably, in compositions or methods for administration of the lipid nanoparticle of the disclosure to a subject, the lipid nanoparticle is combined with a pharmaceutically acceptable carrier as is understood in the art. Accordingly, one example of the present disclosure provides a composition (e.g., a pharmaceutical composition) comprising the lipid nanoparticle of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A lipid nanoparticle of the present disclosure is useful for parenteral, topical, oral, or local administration, intramuscular administration, aerosol administration, or transdermal administration, for prophylactic or for therapeutic treatment. In one example, the lipid nanoparticle is administered parenterally, such as intramuscularly, subcutaneously or intravenously. For example, the lipid nanoparticle is administered intramuscularly.

Formulation of lipid nanoparticle to be administered will vary according to the route of administration and formulation (e.g., solution, emulsion, capsule) selected. An appropriate pharmaceutical composition comprising a lipid nanoparticle to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The lipid nanoparticle can be stored in the liquid stage or can be lyophilized for storage and reconstituted in a suitable carrier prior to use according to art-known lyophilization and reconstitution techniques.

The optimum concentration of the active ingredient(s) (i.e., the RNA) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the lipid nanoparticle of the disclosure are those large enough to produce the desired effect. For example, the composition comprises an effective amount of the encapsulated

RNA. In one example, the composition comprises a therapeutically effective amount of the RNA. In another example, the composition comprises a prophylactically effective amount of the RNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

### RNA

The present disclosure provides a lipid nanoparticle for delivery of RNA, wherein a nucleic acid-binding protein or peptide is bound to the RNA. For example, the present disclosure provides a lipid nanoparticle for delivery of RNA, wherein a lipidated nucleic acid-binding protein or peptide is bound to the RNA.

The present disclosure provides a lipid nanoparticle for delivery of RNA, wherein a RNA-binding protein or peptide is bound to the RNA. For example, the present disclosure provides a lipid nanoparticle for delivery of RNA, wherein a lipidated RNA-binding protein or peptide is bound to the RNA.

The RNA of the present disclosure may be a naturally or non-naturally occurring RNA, or may include one or more modified nucleobases, nucleosides, or nucleotides. It will be apparent to the skilled person that RNA suitable for use in the present disclosure may also include a 5′ untranslated region (5′-UTR), a 3′ untranslated region (3′UTR), and/or a coding or translating sequence. In addition, the RNA may comprise a 5′ cap structure, a chain terminating nucleotide, a stem loop (e.g., a histone stem loop), a 3′ tailing sequence (e.g., a polyadenylation signal or one or more poly A tails.

In one example of the present disclosure, the RNA is a self-replicating mRNA (sa-mRNA).

In one example of the present disclosure, the RNA is a conventional mRNA (cRNA).

### Methods of Preparation

Suitable methods for the production of a lipid nanoparticle of the present disclosure will be apparent to the skilled person and/or described herein. For example, a lipid nanoparticle of the present disclosure may be made using approaches which are well-known in the art of formulation. For example, suitable LNPs can be formed using mixing processes such as microfluidics, including herringbone micromixing, and T-junction mixing of two fluid streams, one of which contains the messenger RNA, typically in an aqueous solution, and the other of which has the various required lipid components, typically in ethanol.

The LNPs may then be prepared by combining a phospholipid (such as DOPE or DSPC, which may be purchased from commercial sources including Avanti Polar Lipids, Alabaster, AL), a PEGylated lipid (such as 1,2-dimyristoyl-sn-glycerol methoxypoly ethylene glycol, also known as PEG-DMG, which may be purchased from commercial sources including Avanti Polar Lipids, Alabaster, AL), and a structural lipid/sterol (such as cholesterol, which may be purchased from commercial sources including Sigma-Aldrich), at concentrations of, for example, about 50 mM in ethanol. Solutions should be refrigerated during storage at, for example, −20° C. The various lipids may be combined to yield the desired molar ratios and diluted with water and ethanol to a final desired lipid concentration of, for example, between about 5.5 mM and about 25 mM.

An LNP composition comprising a RNA, including, but not limited to, as a sa-mRNA or cRNA, may prepared by combining the above lipid solution with a solution including the RNA at, for example, a lipid component to RNA wt: wt ratio from about 5:1 to about 50:1. The lipid solution may be rapidly injected using a NanoAssemblr microfluidic system at flow rates between about 3 ml/min and about 18 ml/min into the RNA solution to produce a suspension with a water to ethanol ratio between about 1:1 and about 4:1.

For LNP compositions including a sa-mRNA or cRNA, solutions of the RNA at concentrations of 1.0 mg/ml in deionized water may be diluted in 50 mM sodium citrate buffer at a pH between 3 and 6 to form a stock solution.

LNP compositions may be further processed, as is known in the art, by 10-fold dilution into 50 mM citrate buffer at pH 6 and subjected to tangential flow filtration (TFF) using a 300k molecular weight cut-off membrane (mPES) until concentrated to the original volume. Subsequently, in one example, the citrate buffer may be replaced with a buffer containing 20 mM Tris buffer at pH 7.5, 80 mM sodium chloride, and 3% sucrose using diafiltration with a 10-fold volume of the new buffer. The LNP solution may be concentrated to a volume of, for example, between 5-10 mL, filtered using a 0.2 micron PES syringe filter, aliquoted into vials, and frozen at 1° C./min using a Corning® CoolCell® LX Cell Freezing Container until the samples reach −80° C. Samples may be stored at −80° C. until needed.

The method described above induces nano-precipitation and particle formation. Alternative processes including, but not limited to, T-junction and direct injection, may be used to achieve the same nano-precipitation.

In some embodiments, the lipid component of the LNP formulation comprises about 2 mol % to about 25 mol % phospholipid (neutral lipid), about 18.5 mol % to about 60 mol % structural lipid (sterol), and about 0.2 mol % to about 10 mol % of PEGylated lipid, provided that the total mol % does not exceed 100%. In some embodiments, the lipid component of the LNP formulation comprises about 5 mol % to about 20 mol % phospholipid, about 30 mol % to about 55 mol % structural lipid, and about 1 mol % to about 5 mol % of PEGylated lipid. In a particular embodiment, the lipid component includes about 10 mol % phospholipid, about 48 mol % structural lipid, and about 2.0 mol % of PEG lipid. In some embodiments, the phospholipid may be DOPE or DSPC. In other embodiments, the PEG lipid may be PEG-DMG and/or the structural lipid may be cholesterol.

The efficiency of encapsulation of the RNA within the LNPs may be at least 50%, for example about 50%, 55%, 60%, 65%, 70%, 75%, 80%, 85%, 90%, 91%, 92%, 93%, 94%, 95%, 96%, 97%, 98%, or 99%. In some embodiments, the encapsulation efficiency may be at least 80%. In certain embodiments, the encapsulation efficiency may be at least 90%.

### Assaying Lipid Nanoparticles of the Disclosure

Lipid nanoparticles of the present disclosure are readily screened for physical and biological activity and/or stability using methods known in the art and/or as described below.

Assessing RNA degradation

In one example, the level of RNA degradation by RNases is assessed. For example, the RNA, alone or in combination with the RNA-binding protein or peptide, is treated with RNase.

In one example, the level of RNA is assessed in RNAse treated and untreated samples using real time PCR. In one example, the cycle threshold (CT) value in RNA samples without a RNA-binding protein or peptide are increased compared to RNA samples with a RNA-binding protein or peptide indicating RNA degradation.

Assessing RNA translation

In one example, RNA translation is assessed using an in vitro translation system. Suitable systems for use in the present disclosure will be apparent to the skilled person, and include for example a rabbit reticulocyte lysate assay.

In one example, a rabbit reticulocyte lysate assay is used.

In one example, the RNA is assessed in the presence or absence of a RNA-binding protein or peptide.

In one example, the RNA is nanoluciferase RNA (nLuc RNA) and the amount of RNA translation is measured by the amount of luciferase produced as assessed by measuring luminescence in relative light units (RLU). In one example, the assay is performed at 4° C., 24° C. and/or 37° C. In another example, the assay is performed after incubating the samples for 0 hours, 1 hour, 2 hours, 4 hours, 8 hours, 24 hours, 48 hours or 96 hours.

Assessing TLR induction

In one example, the level of TLR3 and/or TLR8 induction is assessed. For example, the level of TLR3 and/or TLR8 induction by the RNA, alone or in combination with the RNA-binding protein or peptide is assessed. In one example, TLR3 and/or TLR8 induction is assessed using a TLR induction NfKB reporter assay. In this assay, NfKB is operationally linked to a secretary alkaline phosphatase (SEAP). RNA is introduced into either cell type (TLR3 conditionally transduced, or TLR8 conditionally transduced). Binding of the TLR receptor induces NfKB activation and in turn SEAP. In one example, the SEAP level is determined by a chemical reaction and calorific read out.

### EXAMPLES

### Example 1: Protection of RNA from Degradation by RNases

An RNA-free monovalent pooled harvest (MPH) was prepared by treating MPH with RNase.

MPH (B/Malaysia/2506/2004) was treated twice using RNase A (Promega) for 1 hour at 37° C. RNA was extracted from 140 μl treated and untreated MPH (eluted in 60 μl), and tested for haemagglutinin (HA) and neuraminidase (NA) RNA using real time PCR. The results showed no increase in CT value for HA RNA and a small increase for NA RNA (Table 2; Exp. 1A), indicating minimal degradation of viral RNA.

In case the RNase was inactive, the MPH was treated using a different RNase (RNase ONE, Promega M4261), twice, prior to extraction and analysis of RNA. The CT values for both HA and NA RNA increased (Table 2; Exp. 1B), indicating a decrease in the amount of viral RNA present (<83-fold).

Further samples of MPH (180 μl) were treated twice using decreasing doses of RNase ONE in the presence of RNase ONE sample buffer. Analysis of treated samples showed dose-dependent decreases in RNA concentration and increases in CT value (Table 2; Exp. 1C), indicating low levels of degradation of RNA upon treatment of MPH with RNase ONE (<35-fold). Overall, the level of degradation of RNA in treated MPH was unexpectedly small.

MPH (B/Malaysia/2506/2004) was then treated with RNase ONE in the presence or absence of a disruption buffer. Use of the disruption buffer did not increase degradation of viral RNA in MPH treated with RNase (Table 2; Exp. 2).

Treatment of MPH with RNase resulted in small reductions in viral RNA only, indicating possible protection of RNA from degradation by RNases.

TABLE 2
Treatment of MPH with RNase.

| | Volume (μl) | | | | | | | | RNA | | | Fold decrease | |
|---|---|---|---|---|---|---|---|---|---|---|---|---|---|
| | | | Disrupt. | | RNase | | | | conc. | CT value | | HA | NA |
| Experiment | MPH | | buffer | | buffer | | RNase * | | (μg/ml) | HA | NA | RNA | RNA |
| 1A. RNase A | 5 | ml^{#} | —^{#} | | —^{#} | | —^{#} | | NT^{#} | 17.42^{#} | 16.9^{#} | N/A^{#} | N/A^{#} |
| | 5 | ml | — | | — | | 62.5 | | NT | 16.79 | 19.15 | 0 | 4.8 |
| 1B. RNase | 5 | ml | — | | — | | — | | NT | 8.00 | 10.00 | N/A | N/A |
| ONE | 5 | ml | — | | 0.5 | ml | 1 | μl | NT | 12.46 | 16.36 | 22.0 | 82.1 |
| 1C. RNase | 180 | μl^{#} | —^{#} | | 20 | μl^{#} | —^{#} | | 123^{#} | 8.79^{#} | 8.85^{#} | N/A^{#} | N/A^{#} |
| ONE | 180 | μl | — | | 20 | μl | 0.5 | μl | 95.4 | 10.5 | 9.97 | 3.3 | 2.2 |
| | 180 | μl | — | | 20 | μl | 1 | μl | 71.3 | 11.39 | 10.75 | 6.1 | 3.7 |
| | 180 | μl | — | | 20 | μl | 5 | μl | 56.6 | 12.05 | 11.76 | 9.6 | 7.5 |
| | 180 | μl | — | | 20 | μl | 10 | μl | 51.7 | 13.24 | 13.97 | 21.9 | 34.8 |
| 2. RNase | 250 | μl | 50 | μl | 50 | μl | 10 | μl | 116.2 | 14.00 | 14.42 | 0 | 0 |

| | | | | | | | | | | | | |
|---|---|---|---|---|---|---|---|---|---|---|---|---|
| ONE ** | 250 | μl | 50 | μl | — | 10 | μl | 113.4 | 14.03 | 14.40 | 0 | 0 |

| | | | | | | | | | | |
|---|---|---|---|---|---|---|---|---|---|---|
| 250 | μl | — | — | 10 | μl | 69.1 | 16.03 | 15.42 | 3.7 | 1.9 |

| | | | | | | | | | | |
|---|---|---|---|---|---|---|---|---|---|---|
| 250 | μl | 50 | μl | — | — | 117.3 | 14.01 | 14.44 | 0 | 0 |

| | | | | | | | | | |
|---|---|---|---|---|---|---|---|---|---|
| 250 | μl^{#} | —^{#} | —^{#} | —^{#} | 101.2^{#} | 14.15^{#} | 14.50^{#} | N/A^{#} | N/A^{#} |

* All RNA treatment was performed twice, each for 1 h at 37° C.
NT—not tested.
** Volumes were equalised using PBS.
^{#}Baseline level

### Example 2: Purification of RNA-Free Nucleoprotein from Influenza Virus

RNA-free nucleoprotein (NP) from influenza virus was isolated by performing dialysis, concentration and detergent treatment followed by a glycerol step gradient centrifugation to isolate the NP:RNA particles. To isolate the RNA-free NP, a further glycerol and caesium chloride gradient centrifugation step was performed.

### Example 3: RNA-Free NP Binds RNA

To assess whether RNA-free NP protects RNA, NP was combined with nanoluciferase mRNA and samples analysed by MOPS-Agarose Gel electrophoresis following heating to 40° C. or incubation at room temperature. On increasing the concentration of NP in the reaction (from 0 to 4000 ng), whilst maintaining the concentration of mRNA (250 ng), the level of detectable mRNA shifted to a higher molecular weight.

At a ratio of 16:1 (4000 ng NP; 250 ng mRNA), the nanoluciferase mRNA was shifted to be unable to enter the gel. These results indicate that in the presence of increasing NP concentration, increasing amounts of NP can be complexed to the RNA.

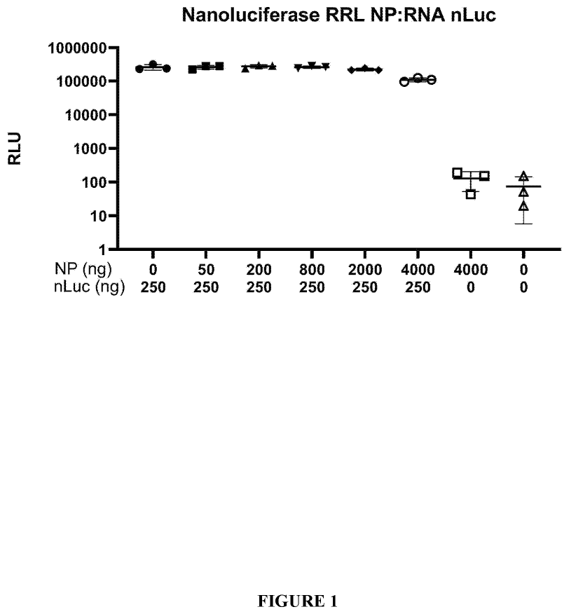

Using a rabbit reticulocyte lysate as an in vitro translation system, it was shown that at a ratio of 16:1 (4000 ng NP; 250 ng mRNA), the amount of luciferase produced as assessed by measuring luminescence in relative light units (RLU) was comparable to luciferase RNA alone, whilst 4000 ng of NP alone reduced the signal by 2-3 fold (FIG. 1). These results indicate that in the presence of increasing NP concentration, RNA is bound by the NP without inhibiting in vitro translation.

### Example 4: RNA-Free NP Protects RNA from Degradation

To assess whether RNA-free NP protects RNA, the NP:RNA and RNA alone were assessed in a RNase assay. Briefly, NP:RNA or RNA was treated with RNase and incubated for 5-10 minutes at 30° C. Samples were further treated with or without lul thermolabile proteinase K (PK; NEB P8111S). The reaction was incubated at 37° C. for 15-30 minutes, followed by incubation at 60° C. for 10-20 minutes to inactivate the PK. 1-2 μl RNasine (Promega N2611) was added if required. The level of RNA present was assessed in treated and untreated samples using real time PCR.

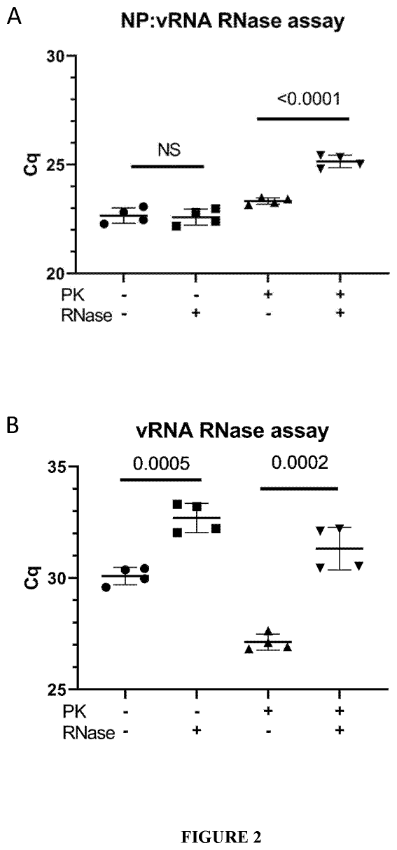

As shown in FIG. 2, there is no increase in CT value in NP:RNA treated with RNase indicating no degradation of viral RNA. In contrast, the CT value of RNA treated with RNase alone significantly increased (p=0.0005), indicating a decrease in the amount of viral RNA present. These results further confirm that in the presence of NP, RNA is protected from degradation by RNase.

The addition of PK, which degrades NP, to NP:RNA showed a slight increase in CT value indicating minimal degradation of RNA. The addition of PK in combination with RNase showed a significant increase in CT value (<0.0001) indicating a decrease in the amount of viral RNA present. These results further confirm that in the presence of NP, RNA is protected from degradation by RNase.

### Example 5: RNA-Free NP Protects RNA from Degradation at 4° C., 24° C. and 37° C.

To assess whether the ability of NP to protect nanoluciferase RNA (nLuc RNA) was temperature dependent, NP: nLuc RNA and nLuc RNA was incubated at 4° C., 24° C. and 37° C. for up to 96 hours and the amount of luciferase produced was assessed by measuring luminescence in RLU.

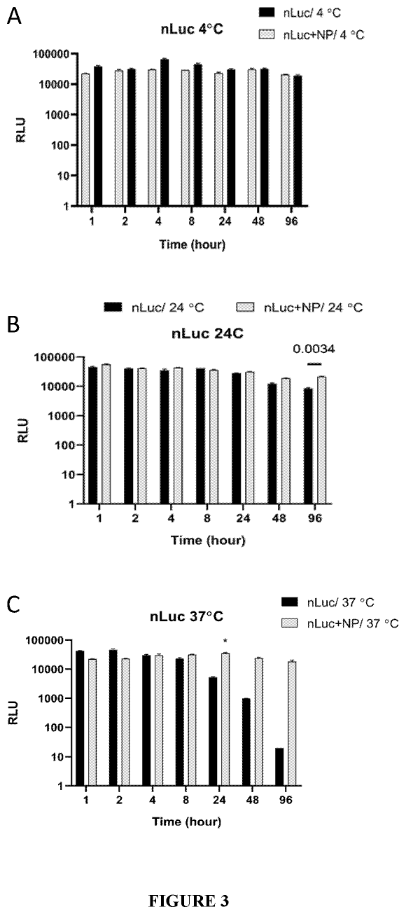

As shown in FIG. 3, there was no significant difference in RLU between NP: nLuc RNA and nLuc RNA alone at 4° C. At 24° C., there was a notable reduction in RLU at 96 hours post incubation (p=0.0034) in nLuc RNA compared to NP: nLuc RNA indicating protection of the RNA from degradation by the NP. After 8 hours at 37° C., there was a notable reduction in RLU in nLuc RNA compared to NP: nLuc RNA, demonstrating further long term protection of the RNA by the NP.

### Example 6: RNA-Free NP Protects TLR Induction

To assess whether the presence of NP inhibits RNA induction of dsRNA or ss RNA, TLR3 and/or TLR8 induction, a TLR induction NfKB reporter assay was used. In this assay, NfKB is operationally linked to a secretary alkaline phosphatase (SEAP). RNA is introduced into either cell type (TLR3 conditionally transduced, or TLR8 conditionally transduced). Binding of the TLR receptor induces NfKB activation and in turn SEAP. The SEAP level can be determined by a chemical reaction and calorific read out.

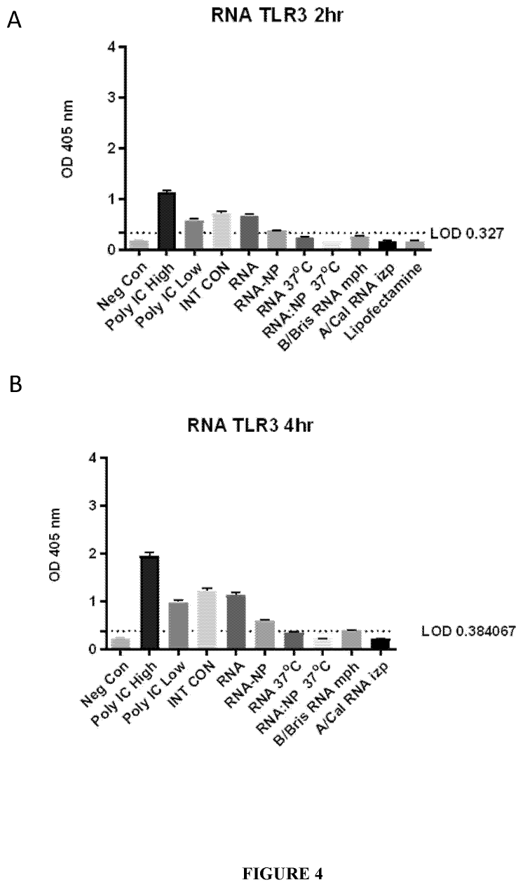

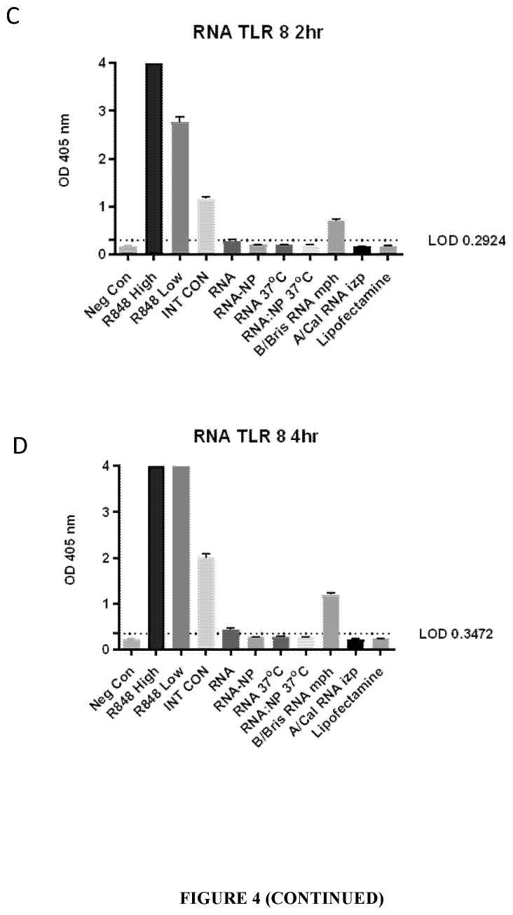

As shown in FIG. 4, RNA alone stimulates both TLR3 and TLR8 induction and the presence of NP reduces the induction of TLR3 and TLR8 at both 2 and 4 hours post-incubation.

### Example 7: RNA-Free NP Conjugation with Maleimide-DSPE Lipid

NP (0.85 mg/ml) was conjugated with maleimide-DSPE (1,2-Distearoyl-sn-glycero-3-phosphorylethanolamine; 8 mM) by incubating NP with DSPE in the presence of 10% ethanol. The labelled and unlabelled NP was run on a non-reducing gel to assess molecular weight of the protein and confirm conjugation of the protein with a molecular weight shift of the NP. In addition, DSPE forms a suspension (i.e., liposomes) so the composition was centrifuged and the supernatant assessed.

It was subsequently shown that lipidated NP:RNA could be pulled down by centrifugation, whilst RNA alone in the presence of lipid (i.e., not in the presence of NP) could not be.

### Example 8: COVID Nucleocapsid Protects RNA

RNA-free nucleocapsid from SARS-CoV-2 (NP (SCOV2)) was purified as described above for influenza virus. The ability of NP (SCoV2) to protect nLuc RNA from degradation was assessed in an in vitro translation system by measuring the amount of luciferase produced as assessed by measuring luminescence in RLU for up to 168 hours.

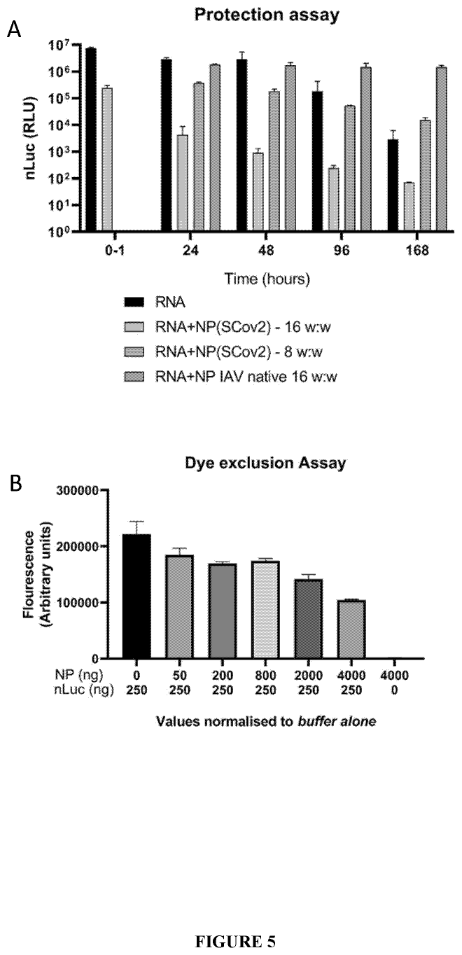

As shown in FIG. 5, there was a reduction in RLU in nLuc RNA alone over time indicating degradation of the nLuc RNA. Whilst there appeared to be a slight reduction in RLU in NP (SCoV2):nLuc RNA compositions at 16:1 and 8:1 (w/w), suggesting RNA degradation, PCR analysis of the NP (SCoV2):nLuc RNA material at each time point confirmed that the RNA is intact and not degraded.

In addition, the accessibility of the NP (SCoV2) encapsulated RNA was assessed using a ribogreen dye exclusion assay. As shown in FIG. 5B, the presence of the NP (SCoV2) at increasing concentrations interferes with the dye binding to the nLuc RNA, however based on the fluorescence signal, the RNA is still accessible to the dye at a 16:1 ratio (4000 ng NP; 250 ng mRNA). This suggests that whilst the NP (SCoV2) coats and protects the RNA, it does not completely encapsulate the RNA.

### Example 9: Preparation of RNA-Binding Peptides

Cellular and viral protein sequences were reviewed to identify protein domains and peptide sequences that had the potential to bind RNA. Sequences from cellular proteins correlate to those proteins associated with cell growth, cell signalling and/or antiviral pathways whereas sequences from viral proteins were derived from non-structural and nuclear proteins.

Peptides were designed from sequences derived from cellular proteins including TAR RNA binding protein (TRBP), Protein Kinase R (PKR), Toll-like receptor 3 (TLR-3) and TLR-7 (Table 3). Viral RNA binding proteins included nucleoprotein and non-structural proteins from influenza (Table 4).

RNA binding peptide sequences were modified to either exclude known nuclear localisation signals, or include nuclear export signals to facilitate correct localisation of peptide bound RNA when this material is introduced into cells (Table 3).

TABLE 3
RNA binding peptides derived from cellular proteins

| Peptide | SEQ | | | |
|---|---|---|---|---|
| # | ID NO | Name | Sequence | Description |
| 1 | 1 | TRBP RBD-A | VGALQELVVQKGWRLPEYTVTQESGPAHRKEFTMTCRVERFIE | TRBP RNA binding |
| | | | IGSGTSKKLAKRNAAAKMLLRV | domain 2 (full) |
| 2 | 2 | TRBP RBD-B | SGPAHRKEFTMTCRVERFIEIGSGTSKKLAKRNAAAKMLLRV | TRBP RNA binding |
| | | | | domain 2 (truncated) |
| 3 | 3 | PKR RBM-A | NYIGLINRIAQKKRLTVNYEQCASGVHGPEGFHYKCKMGQKEY | PKR RNA binding |
| | | | SIGTGSTKQEAKQLAAKLAYQILSE | motif 2 |
| 4 | 4 | PKR RBM-B | NYIGLINRIAQKKRLTVNYEQCASGVHGPEGFHYKCKMGQKEY | PKR RNA binding motif 2 |
| | | | | (truncated-1) |
| 5 | 5 | PKR RBM-C | SGVHGPEGFHYKCKMGQKEYSIGTGSTKQEAKQLAAKLAYQIL | PKR RNA binding motif 2 |
| | | | SE | (truncated-2) |
| 6 | 6 | TLR-3 LRR-A | NITVLNLTHNQLRRLPAANFTRYSQLTSLDVGFNTISKLEPEL | TLR-3 dsRNA binding |
| | | | CQKLPMLKVLNLQHNELSQLSDKTFAF | domain 1 (LRR 1-3) |
| 7 | 7 | TLR-3 LRR-B | YNKYLQLTRNSFALVPSLQRLMLRRVALKNVDSSPSPFQPLRN | TLR-3 dsRNA binding |
| | | | LTILDLSNNNIANINDDMLEG | domain 1 (LRR 17-18) |
| 8 | 8 | TLR-7 LRR-A | LQNLEVLDLGTNFIKIANLSMFKQFKRLKVIDLSVNKISPSGDS | TLR-7 RNA binding site |
| | | | SEVG | (LRR 14-15) |

TABLE 4
Viral RNA binding proteins

| Peptide | SEQ | | | |
|---|---|---|---|---|
| # | ID NO | Name | Sequence | Description |
| 1 | 9 | NS RBD-A | GATNATINFEAGILECYERLSWQRALDYPGQDRLNRLKRK | Influenza B NS1 RNA binding |
| | | | LESRIKTHNKSEPESKRMSLEERKAIGVKMMKVLL | domain (3 helices) |
| 2 | 10 | NS RBD-B | ESRIKTHNKSEPESKRMSLEERKAIGVKMMKVLL | Influenza B NS1 RNA binding |
| | | | | domain truncated |
| 3 | 11 and | NS RBD-C | MANNNMTTTQIEWRMKKMAIGSSTHSSS-linker- | Modified Influenza B NS1 RNA |
| | 10 | | ESRIKTHNKSEPESKRMSLEERKAIGVKMMKVLL | binding domain |

### Example 10: Protection of RNA from Degradation in the Presence of RNA-Binding Peptides

To assess whether RNA binding peptides (RBP) protect RNA, RBP:RNA and nanoluciferase (nLuc) RNA alone were assessed using real time PCR to illustrate the level of nLuc RNA remaining following RNase treatment. The RBP assessed were TAR RNA binding protein (TRBP) domains A and B, Protein Kinase R (PKR) domains A and B, influenza non-structural protein (NS RBD) domains A, B and C, Toll-like receptor 7 (TLR-7) an TLR-7.

Briefly, RBP:RNA samples were prepared by combining RBP and NLuc RNA and incubating for 1 hour at 37° C. Then samples of the RBP:RNA and NLuc RNA were prepared with and without RNase (Promega M426A). The samples treated with RNase were incubated for 30 minutes at room temperature. The level of RNA present was assessed in treated and untreated samples using real time PCR.

Real time PCR was performed in the presence of SYBR-green (QuantiNova SYBR green RT-PCR kit #208152).

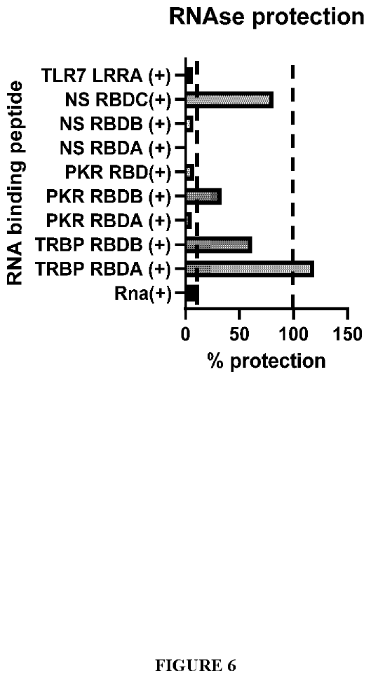

CT values were compared (Table 5) and the percentage of protection calculated. As shown in FIG. 6, NS RBDC, TRBP RBDA and TRBP RBDB demonstrated a level of protection for RNA from RNase degradation. The remaining binding domains demonstrated residual RNA below the level of non-peptide bound RNA.

TABLE 5
Cycle threshold (CT) values

| Sample | | Ct value | CT value |
|---|---|---|---|
| number | Peptide | No RNase | Plus RNase |
| Sample 1 | TRBP RBD-A | 20.4 | 20.1 |
| Sample 2 | TRBP RBD-B | 20.0 | 20.7 |
| Sample 3 | PKR RBM-A | 14.6 | 18.8 |
| Sample 4 | PKR RBM-B | 17.9 | 19.5 |
| Sample 5 | PKR RBM-C | 16.9 | 20.6 |
| Sample 6 | NS RBD-A | 13.2 | 20.5 |
| Sample 7 | NS RBD-B | 14.8 | 18.6 |
| Sample 8 | NS RBD-C | 17.5 | 17.8 |
| Sample 9 | TLR-3 LRR-A | Undetermined | 14.1 |
| Sample 10 | TLR-3 LRR-B | Undetermined | Undetermined |
| Sample 11 | TLR-7 LRR-A | 15.0 | 19.0 |
| Sample 12 | RNA alone | 11.0 | 14.0 |
| Water | | | 31.6 |

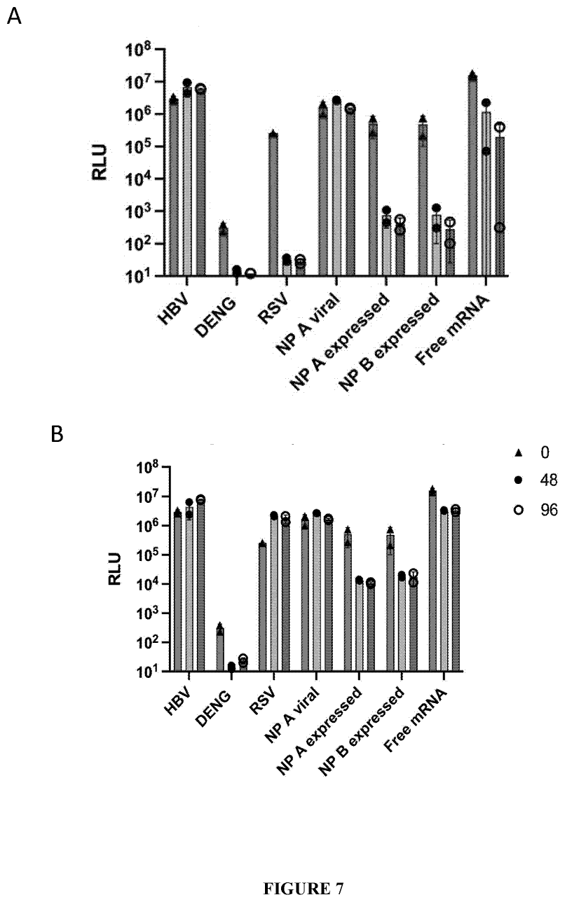

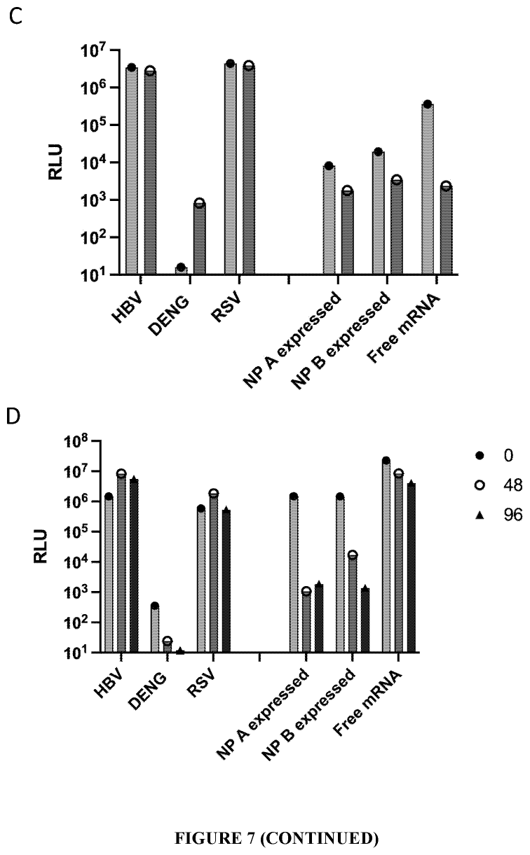

HBV, Dengue, RSV, influenza A were also assessed (FIG. 7). The data demonstrates that the HBV binding protects RNA when RNase is added. However, recombinant Dengue does not allow transcription similar to RSV which is non-protective. The recombinant influenza A and B NP are only partially protective.

### Example 11: RNA: NP In Vivo Expression

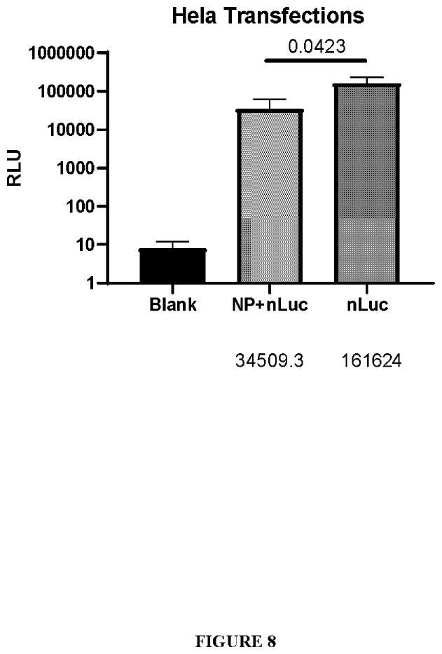

To assess whether NP:RNA can be translated in cells, Hela cells were transfected with NP:RNA or nLuc RNA alone. Briefly, 10 ng/well NP:RNA or nLuc RNA was added to cells with Lipofectamine 3000. Cells were washed 2 hours post-transfection and the amount of nLuc produced was assessed by measuring luminescence in RLU at 24 hours post transfection. As shown in FIG. 8, NP:RNA are expressed in Hela cells.

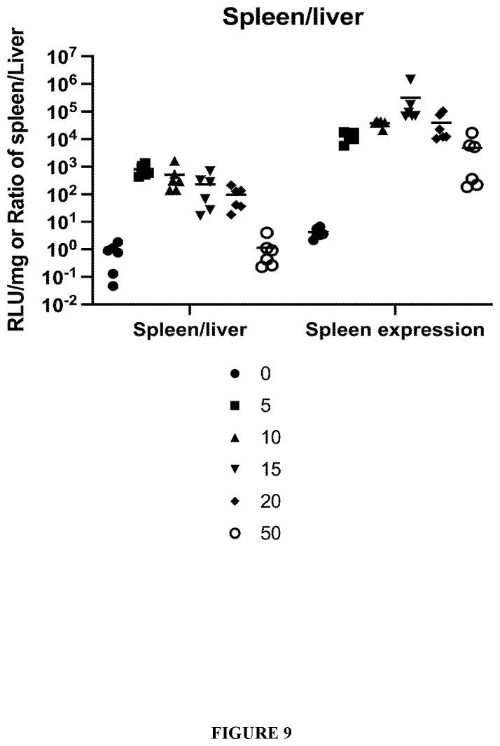

nLuc RNA expression was also measured in the spleen and liver as shown in FIG. 9.

### Example 12: Formulation of NP:mRNA LNPs

LNPs were prepared with and without RNA bound to nucleoprotein. In order to determine whether the nucleoprotein was completely encapsulated, various LNP formulations were developed (Table 6) and analysed for average size (Z-Ave) and polydispersity index (PDI) (Table 7). The LNPs were developed with or without nucleoprotein, and RNA was placed internally (interior) or on the surface of the LNP (exterior).

TABLE 6
LNP Formulation ratios

| Formulation Name | MC3:DSPC:Chol:PEG ratio |
|---|---|
| 0:1.5 | 0:39.4:59.1:1.5 |
| 5:1.5 | 5:37.4:56.1:1.5 |
| 10:1.5 | 10:35.4:53.1:1.5 |
| 15:1.5 | 15:33.4:50.1:1.5 |
| 20:1.5 | 20:31.5:47:1.5 |
| 50:1.5 | 50:10:38.5:1.5 |

TABLE 7
LNP Formulation

| LNP formulation | Z-Ave | |
|---|---|---|
| Experiment 1 | (d.mn) | PdI |
| interior DOTAP-LNP | 84.22 | 0.359 |
| interior DOTAP-LNP (NP + nLuc) | 125.1 | 0.231 |
| exterior DOTAP-LNP (nLuc) | 87.66 | 0.242 |
| exterior DOTAP-LNP (NP + nLuc) | 137.1 | 0.147 |
| Experiment 2 | Z-Ave | PdI |
| empty DOTAP-LNP 1 | 79.55 | 0.422 |
| interior DOTAP-LNP (nluc) 1 | 83.01 | 0.387 |
| interior DOTAP-LNP (NP + nluc) 1 | 168.8 | 0.141 |
| exterior DOTAP-LNP (nluc) 1 | 78.05 | 0.287 |
| exterior DOTAP-LNP (NP + nluc) 1 | 184.9 | 0.156 |

### Example 13: NP:LNP Thermostability

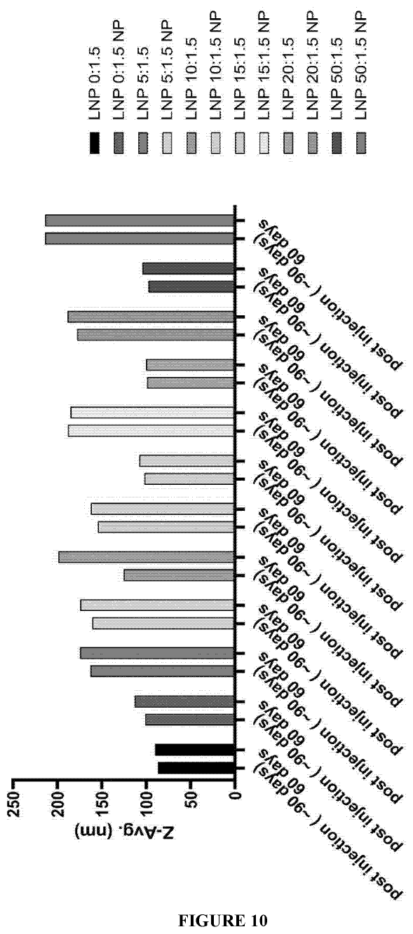

To assess the stability of NP:LNP formulations over time, the LNPs were assessed 60 and 90 days post injection at 4° C. by determining the Z-average diameter of the LNPs. As shown in FIG. 10, NP containing LNPs were thermostable over time at 4° C.

### Example 14: In Vivo Biodistribution of LNP mRNA

To assess the biodistribution of the 0:1.5, 15:1.5 and 50:1 LNP formulations with and without NP were injected intramuscular into mice and then the organ biodistribution at day 6 post injection was assessed by measuring the amount of luminescence signal (RLU) per mg of each organ.

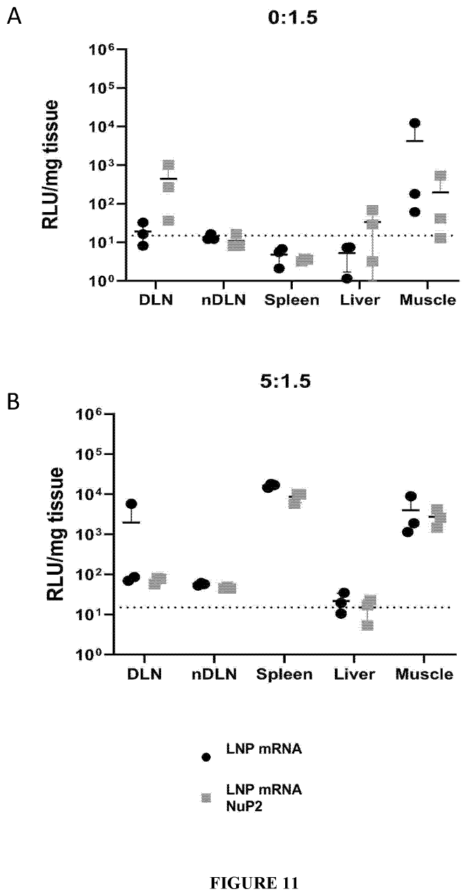

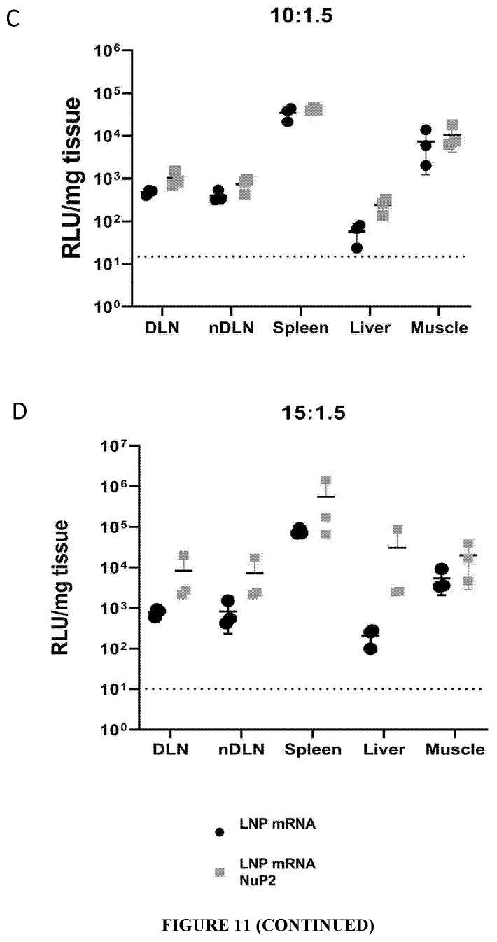

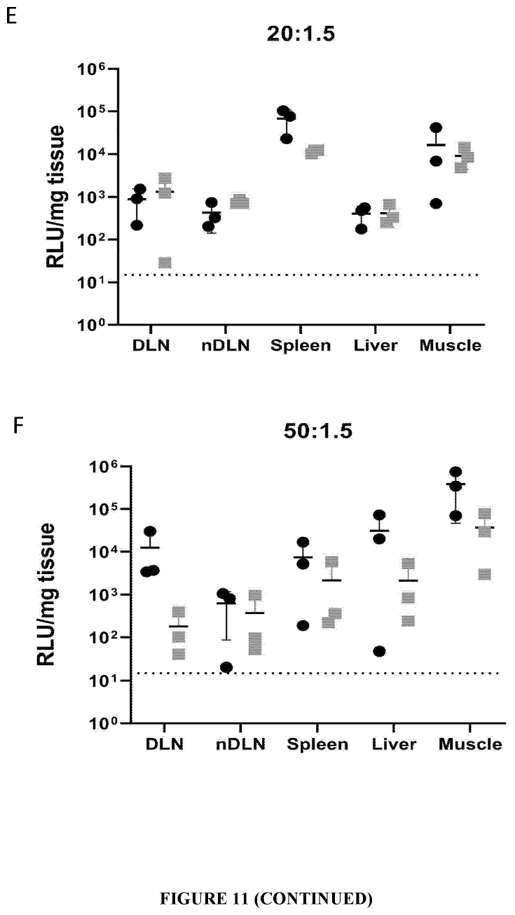

As shown in FIG. 11, LNPs formulated with NP:mRNA or nLuc mRNA alone were detected in draining and non-draining lymph nodes, spleen, liver and muscle. Increased RLU was observed in the draining and non-draining lymph node and liver in mice receiving 15:1.5 NP-mRNA-LNP formulations when compared to mRNA-LNP.

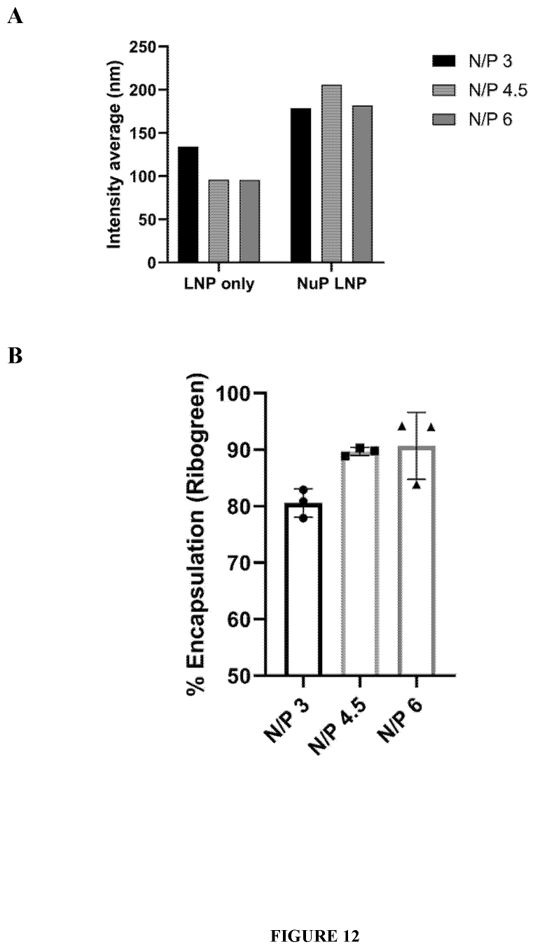

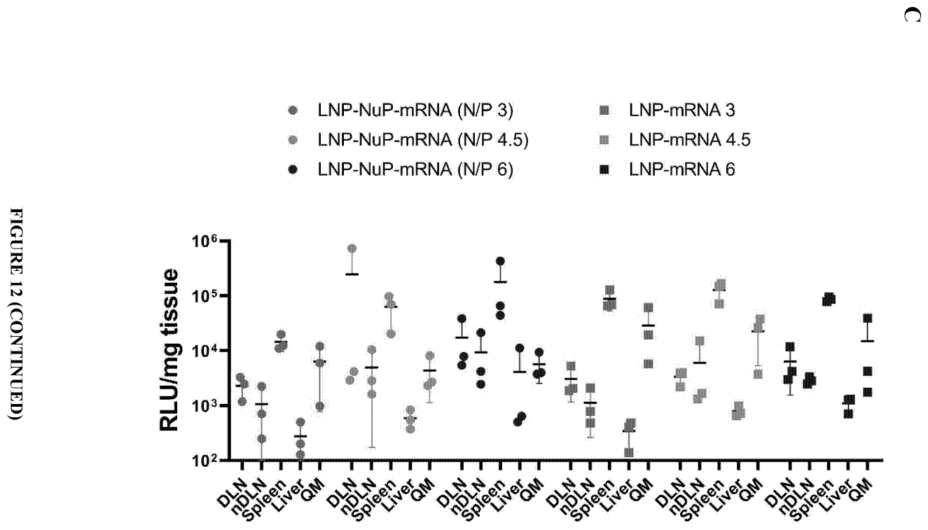

The effect of altering the nitrogen (N) to phosphate (P) ratio (N/P) in the LNP was also assessed. As shown in FIG. 12, the effect of a N/P ratio of 3, 4.5 and 6 was assessed on LNP size (A) and encapsulation efficiency (B). The effect on biodistribution was also assessed and the results are shown in FIG. 12C. Similar biodistribution for LNP-mRNA and NuP-LNP-mRNA in all other organs at all N/P ratios (3, 4.5 and 6) was observed.

1. A lipid nanoparticle for delivery of RNA, the lipid nanoparticle comprising therein:
(i)_a nucleic acid-binding protein or peptide bound to the RNA, or
(ii) an RNA-binding protein or peptide bound to the RNA.

2. The lipid nanoparticle of claim 1, wherein:
(i) the nucleic acid-binding protein or peptide is an RNA-binding protein or peptide, or
(ii) the nucleic acid-binding protein or peptide is a lipidated nucleic acid-binding protein or peptide bound to the RNA, or
(iii) the RNA-binding protein or peptide is a lipidated RNA-binding protein or peptide.

3. (canceled)

4. (canceled)

5. A lipid nanoparticle for delivery of RNA, the lipid nanoparticle comprising therein:
(i) a lipidated nucleic acid-binding protein or peptide bound to the RNA, or
(ii) a lipidated RNA-binding protein or peptide bound to the RNA.

6. (canceled)

7. The lipid nanoparticle of claim 2, wherein the RNA-binding protein or peptide is lipidated:
(a) prior to binding the RNA, and/or
(b) with a lipid moiety selected from the group consisting of a fatty acid, an isoprenoid and combinations thereof, and/or
(c) on a nucleophilic side chain, at the N-terminal end and/or at the C-terminal end, and/or
(d) by palmitoylation, myristoylation, fatty-acylation, esterification, prenylation, or combinations thereof, and/or
(e) by N-terminal cysteine palmitoylation, N-terminal glycine myristoylation, lysine N-acylation, C-terminal cholesterol esterification, cysteine prenylation, serine O-acylation or combinations thereof, and/or
(f) using chemical or enzymatic lipidation, and/or
(g) using chemical lipidation selected from the group consisting of chemical ligation, click chemistry, expressed protein ligation and combinations thereof, and/or
(h) using enzymatic lipidation selected from the group consisting of Sortase-A mediated lipidation, transglutaminase mediated lipidation and combinations thereof.

8. (canceled)

9. The lipid nanoparticle of claim 7, wherein:
(i) the fatty acid is a triglyceride, a phospholipid or a cholesteryl ester, and/or
(ii) the nucleophilic side chain is a cysteine, a serine, a threonine, a tyrosine and/or a lysine amino acid residue, and/or
(iii) the prenylation is farnesylation or geranylgeranylation.

10.-14. (canceled)

15. The lipid nanoparticle of claim 7, wherein the lipid moiety is linked to the RNA-binding protein or peptide by a thioether bond, an ester bond, a thioester bond and/or an amide bond.

16.-18. (canceled)

19. The lipid nanoparticle of claim 7, wherein the enzymatic lipidation is performed in vivo or in vitro.

20. The lipid nanoparticle of claim 1, wherein the RNA-binding protein or peptide encapsulates the RNA, and/or binds directly to the RNA.

21. (canceled)

22. The lipid nanoparticle of claim 1, wherein the RNA-binding protein or peptide:
a) reduces toxicity of the lipid nanoparticle, and/or
b) stabilizes the RNA, and/or
c) protects the RNA from degradation, and/or
d) facilitates nucleation of the lipid nanoparticle, and/or
e) inhibits induction of signalling by one or more Toll-like Receptors.

23. The lipid nanoparticle of claim 1 [3], wherein the RNA-binding protein or peptide is modified to remove nuclear localisation signal(s) and/or introduce nuclear export signal(s).

24. The lipid nanoparticle of claim 1, wherein the RNA-binding protein or peptide is a viral or non-viral RNA-binding protein or peptide.

25. The lipid nanoparticle of claim 24, wherein the viral RNA-binding protein or peptide:
(a) is from a class III, class IV, class V and/or class VI virus, and/or
(b) is a respiratory virus selected from the group consisting of an influenza virus, a respiratory syncytial virus, a parainfluenza virus, a metapneumovirus, a rhinovirus, a coronavirus, an adenovirus and a bocavirus, and/or
(c) is a nucleoprotein, a non-structural protein, a matrix protein and/or a nucleocapsid protein, and/or
(d) is a non-structural (NS) protein from an influenza B virus, and/or
(e) comprises a sequence set forth in any one of SEQ ID NOs: 9 to 11.

26.-29. (canceled)

30. The lipid nanoparticle of claim 24, wherein the non-viral RNA binding protein or peptide is derived from a cellular protein associated with cell growth, cell signalling and/or anti-viral pathways.

31. The lipid nanoparticle of claim 30, wherein the cellular protein:
(i) is selected from the group consisting of a TAR RNA-binding protein (TRBP), a protein kinase R (PKR) RNA-binding protein, a Toll-like Receptor 3 (TLR-3) binding protein, a TLR-7 binding protein and combinations thereof, and/or
(ii) comprises a sequence set forth in any one of SEQ ID NOs: 1 to 8.

32. (canceled)

33. The lipid nanoparticle of claim 1, wherein the lipid nanoparticle:
(a) additionally comprises a PEG-lipid, a structural lipid, an ionisable lipid and/or a neutral lipid, and/or
(b) does not comprise a cationic lipid.

34. (canceled)

35. The lipid nanoparticle of claim 1, wherein the RNA is selected from the group consisting of messenger RNA (mRNA), small-interfering RNA (siRNA), microRNA (miRNA) and antisense RNA.

36. The lipid nanoparticle of claim 35, wherein the mRNA is self-replicating mRNA (sa-mRNA) or a conventional RNA (cRNA).

37. (canceled)

38. A pharmaceutical composition comprising the lipid nanoparticle of claim 1 and a pharmaceutically acceptable carrier.

39. The pharmaceutical composition of claim 38 for use in therapy.


SEQUENCE LISTING (11 sequences)

SEQ ID NO: 1 - protein, 65 aa, synthetic construct
VGALQELVVQKGWRLPEYTVTQESGPAHRKEFTMTCRVERFIEIGSGTSKKLAKRNAAAKMLLRV

SEQ ID NO: 2 - protein, 42 aa, synthetic construct
SGPAHRKEFTMTCRVERFIEIGSGTSKKLAKRNAAAKMLLRV

SEQ ID NO: 3 - protein, 68 aa, synthetic construct
NYIGLINRIAQKKRLTVNYEQCASGVHGPEGFHYKCKMGQKEYSIGTGSTKQEAKQLAAKLAYQILSE

SEQ ID NO: 4 - protein, 43 aa, synthetic construct
NYIGLINRIAQKKRLTVNYEQCASGVHGPEGFHYKCKMGQKEY

SEQ ID NO: 5 - protein, 45 aa, synthetic construct
SGVHGPEGFHYKCKMGQKEYSIGTGSTKQEAKQLAAKLAYQILSE

SEQ ID NO: 6 - protein, 70 aa, synthetic construct
NITVLNLTHNQLRRLPAANFTRYSQLTSLDVGFNTISKLEPELCQKLPMLKVLNLQHNELSQLSDKTFAF

SEQ ID NO: 7 - protein, 64 aa, synthetic construct
YNKYLQLTRNSFALVPSLQRLMLRRVALKNVDSSPSPFQPLRNLTILDLSNNNIANINDDMLEG

SEQ ID NO: 8 - protein, 48 aa, synthetic construct
LQNLEVLDLGTNFIKIANLSMFKQFKRLKVIDLSVNKISPSGDSSEVG

SEQ ID NO: 9 - protein, 75 aa, synthetic construct
GATNATINFEAGILECYERLSWQRALDYPGQDRLNRLKRKLESRIKTHNKSEPESKRMSLEERKAIGVKMMKVLL

SEQ ID NO: 10 - protein, 34 aa, synthetic construct
ESRIKTHNKSEPESKRMSLEERKAIGVKMMKVLL

SEQ ID NO: 11 - protein, 28 aa, synthetic construct
MANNNMTTTQIEWRMKKMAIGSSTHSSS